(19) United States
(12) Patent Application Publication
(10) Pub. No.: US 2025/0144589 A1
(43) Pub. Date: May 8, 2025

(54) Method for Producing Microcapsules

(71) Applicant: Symrise AG, Holzminden (DE)

(72) Inventors: Ralf BERTRAM, Holzminden (DE); Benjamin ROST, Bodenwerder (DE); Andreas VOGEL, Stadtoldendorf (DE); Britta BLANCHFIELD, Münden (Aller) (DE); Christina KOEPKE, Holzminden (DE); Lisette HOFFARTH, Hamburg (DE); Daniela GREGOR, Bevern (DE)

(21) Appl. No.: 18/835,342

(22) PCT Filed: Feb. 2, 2023
(86) PCT No.: PCT/EP2023/052523

§ 371 Date: Aug. 1, 2024

(30) Foreign Application Priority Data
Feb. 2, 2022 (WO) PCT/EP2022/052501
Mar. 25, 2022 (WO) PCT/EP2022/057952

(51) Int. Cl. B01J 13/14; B01J 13/20

(52) U.S. Cl. CPC B01J 13/14; B01J 13/206

(57) ABSTRACT

The present invention relates to a process for the preparation of microcapsules, in particular plant protein-based microcapsules and dispersions of such microcapsules (microcapsule slurry), which include at least one hydrophobic active ingredient, preferably perfume- or aroma-containing plant protein-based microcapsules, which have a balance of stability and performance compared to microcapsules of the prior art. Furthermore, the present invention relates to plant protein-based microcapsules obtainable by the method according to the invention. In a further aspect, the present invention relates to the use of the plant protein-based microcapsules and dispersions according to the invention as a component of household products, textile care products, detergents, fabric softeners, cleaning agents, scent boosters or fragrance enhancers in liquid or solid form, cosmetics, personal care products, perfume compositions, agricultural products, pharmaceutical products or print coating for paper. Ultimately, the present invention relates to consumer products comprising such microcapsules or microcapsule dispersions according to the invention.

### FIELD OF THE INVENTION

The present invention relates to a process for the preparation of microcapsules, in particular plant protein-based microcapsules and dispersions of such microcapsules (microcapsule slurry), which include at least one hydrophobic active ingredient, preferably perfume- or aroma-containing plant protein-based microcapsules, which have a balance of stability and performance compared to microcapsules of the prior art. Furthermore, the present invention relates to plant protein-based microcapsules obtainable by the method according to the invention. In a further aspect, the present invention relates to the use of the plant protein-based microcapsules and dispersions according to the invention as a component of household products, textile care products, detergents, fabric softeners, cleaning agents, scent boosters or fragrance enhancers in liquid or solid form, cosmetics, personal care products, perfume compositions, agricultural products, pharmaceutical products or print coating for paper. Ultimately, the present invention relates to consumer products comprising such microcapsules or microcapsule dispersions according to the invention.

Microcapsules are particles consisting of a core and a wall material surrounding the core, whereby the core can be a solid, liquid or gaseous substance surrounded by a polymeric dense, permeable or semi-permeable wall material. During production, the polymers from the starting components are deposited on the substances to be encapsulated after emulsification and coacervation or interfacial polymerization. The core is also referred to as the inner phase. Names such as outer phase, shell or coating are also used for the wall. The diameter of the microcapsules typically varies from 1 to 1000 μm. The wall thickness is typically 0.5 to 150 μm, but can be varied in the range from 5·10⁻⁹ m to 5·10⁻⁶ m. Typically, loadings of 25 to 95 wt.-%, but also those of 1 to 99 wt. % are possible.

The encapsulation of an active substance with a suitable wall material (coating material) can generally be carried out for several reasons:
Conversion of liquids into a manageable powder form (e.g. coating of vegetable oils, fats);
Time-controlled release of substances (dosage control, depot effect for pharmaceuticals, pesticides, and fertilizers);
Taste, odor and color lamination (e.g. bitter or pungent flavorings);
Protection from light, oxidation, heat, acids, or bases (e.g. vitamins, flavorings, etc.)
Moisture protection (e.g. hygroscopic salts or minerals);
Delaying the loss of volatile components (e.g. flavorings);
Prevention of premature chemical reactions with other mixture components;
improved handling before or during processing (flow properties, dust formation);
Protection of personnel from harmful or unpleasant materials (chemicals, aroma concentrates); or improved solubility or suspendability through surface modification.

Hydrophobic active ingredients, such as fragrances or flavorings, or fragrances or odiferous substances, respectively, can be easily incorporated into numerous and diverse application formulations through encapsulation.

The contents of microcapsules can generally be released in various ways and are based in particular on one of the mechanisms described below:
The capsules are destroyed mechanically by crushing or shearing. This mechanism is used for carbonless paper, for example.
The capsules are destroyed by melting the wall material. According to this mechanism, ingredients such as raising agents or flavorings in baking mixes are only released during the baking process.
The capsules are destroyed by dissolving the wall material. This mechanism is used in washing powder, for example, so that encapsulated ingredients such as enzymes are only released during the washing process.
The capsules remain intact and the capsule contents are gradually released by diffusion through the capsule wall. This mechanism can be used, for example, to achieve a slow and even release of active pharmaceutical ingredients in the body.

Due to their diverse properties, microcapsules are used in various sectors, including the printing industry, the food industry (vitamins, aromas, plant extracts, enzymes, microorganisms), the agrochemical industry (fertilizers, pesticides), the animal feed industry (minerals, vitamins, enzymes, drugs, microorganisms), the pharmaceutical industry, the detergent industry and the cosmetics industry.

Many everyday articles such as detergents, fabric softeners, washing powders, liquid detergents, shower gels, shampoos, deodorants, poly lotions etc. are nowadays perfumed with fragrances or odiferous substances or mixtures of fragrances or odiferous substances, respectively. Very often, fragrances or odiferous substances interact with other ingredients in the formulation or the more volatile components of a perfume evaporate prematurely. This usually results in the fragrance impression of the perfume changing over time or even disappearing completely.

The microencapsulation of such mixtures of fragrances or odiferous substances offers the possibility of reducing or completely preventing interactions in the perfumed product or the evaporation of the highly volatile fragrance components.

A variety of capsule wall and coating materials are known for the production of microcapsules. The capsule wall can consist of either natural, semi-synthetic or synthetic materials. Natural shell materials are, for example, gum arabic, agar-agar, agarose, maltodextrins, alginic acid or its salts, e.g. sodium alginate or calcium alginate, fats and fatty acids, cetyl alcohol, collagen, chitosan, lecithin, gelatine, albumin, shellac, poly-saccharides such as starch or dextran, polypeptides, protein hydrolysates, sucrose and waxes. Semi-synthetic capsule wall materials include chemically modified celluloses, in particular cellulose esters and cellulose ethers, e.g. cellulose acetate, ethyl cellulose, hydroxypropyl cellulose, hydroxypropyl methyl cellulose and carboxymethyl cellulose, as well as starch derivatives, in particular starch ethers and starch esters. Synthetic shell materials are, for example, polymers such as polyacrylates, polyamides, polyvinyl alcohol or polyvinylpyrrolidone.

Depending on the type of capsule wall material and manufacturing process, microcapsules are produced with different properties in terms of diameter, size distribution and physical and/or chemical properties.

Polyurea microcapsules or polyurea/polyurethane microcapsules formed by polymerization between a polyisocyanate and a polyamine and/or a diol or polyol are well-known capsules used in a variety of technical fields, including perfumery.

Polyurea microcapsules obtained by reacting two polyisocyanates and a polyamine are described, for example, in WO 2011/161229 or WO 2011/160733. According to WO 2011/161229 or WO 2011/160733, the polyurea microcapsules are produced in the presence of polyvinylpyrrolidone (PVP) as a protective colloid. WO 2012/107323 discloses polyurea microcapsules with a polyurea shell comprising the reaction product of a polyisocyanate with guanazole (3,5-diamino)-1,2,4-triazole) and an amino acid in the presence of anionic stabilizers or surfactants such as anionic polyvinyl alcohol. EP 0 537 467 B describes microcapsules prepared from polyisocyanates containing polyethylene oxide groups in the presence of stabilizers such as polyvinyl alcohol. According to WO 2007/096592, microencapsulation can be carried out in an oil phase emulsified in a continuous aqueous phase generally stabilized by a surfactant system such as polyvinyl alcohols or carboxylated and sulfonated derivatives thereof.

The exemplary prior art delivery systems described above exhibit both good stability, namely the ability to retain the active ingredient and thus the ability of the capsules to avoid loss of the volatile components, as well as good performance, for example fragrance or odorant release in the case of fragrance or odorant capsules.

However, the microcapsules of the prior art described above have the disadvantage that the polymeric capsule wall or capsule shell material requires a large proportion of polymer in order to ensure sufficient stability and avoid excessive loss of active ingredients. In addition, microencapsulation introduces plastic into the environment, which can cause problems there as “microplastics”, possibly resulting in environmental damage or adverse health effects.

As plastic particles are increasingly the subject of public criticism with regard to their environmental impact and the demand for bio-based and biodegradable solutions is growing due to increasing social pressure with regard to environmental aspects, there is a need to develop new materials for microencapsulation in order to achieve a reduction in microplastics in the environment. The focus here is on bio-based and biodegradable materials.

Against this backdrop, there is therefore a need to provide microcapsules that are increasingly manufactured using biodegradable capsule wall materials that also have outstanding stability and excellent release properties for the respective applications. It is important that not only the macromolecular material of the capsule wall itself is biocompatible, but also each of the fragments produced during disintegration. The aim is also to address consumer groups who wish to avoid animal-based ingredients in the end products.

However, this task of reducing the amount of microplastics in the environment with the help of biodegradable materials is not trivial in the case of microencapsulations, as the desired functionality of the microcapsule, such as olfactory properties and positive secondary properties such as high stability and toxicological harmlessness, conflicts with the requirements for rapid biodegradability in many applications.

It is particularly difficult to produce microcapsules that have both good stability and good drug release. The ability to retain the active ingredient and thus the ability of the capsules to avoid the loss of the volatile components depends in particular on the stability of the capsules in the product base. However, capsules with good stability in particular do not automatically have good biodegradability.

As the degree of cross-linking increases, the stability of the microcapsules increases, but at the same time the ability of the capsule shell to biodegrade decreases. Very stable microcapsules have a lower performance, for example sensory performance, as the number of microcapsules that break open due to pressure, friction, etc. and release active ingredients decreases. If they are too unstable, they are destroyed during storage and also fail to perform.

Recently, the demand for vegan products has also increased, which can be attributed to an increase in consumer awareness, among other things. Veganism is a diet and lifestyle that emerged from vegetarianism, whereby vegans are known to avoid all foods of animal origin. In connection with veganism, corresponding arguments from the areas of animal ethics, environmental protection, world hunger, health and religion must also be taken into account. As veganism is also expanding into other areas of daily life and needs, there is an increased demand for vegan microcapsules that cover the above-mentioned advantages and areas of application. For the purposes of the present invention, vegan microcapsules can be understood and described as plant protein-based microcapsules. They are free of any animal components, in particular animal protein components. This can also be understood and described as meaning that the plant protein-based microcapsules according to the invention are free of protein components of animal origin.

In particular, the present invention is to be understood and described in such a way that plant protein-based microcapsules, i.e. vegan microcapsules, do not contain any animal protein components or any other components of animal origin. A plant protein-based microcapsule according to the present invention thus consists exclusively of proteins or protein components of plant origin with regard to the proteins used. Animal proteins or protein components, for example gelatine, are explicitly excluded with regard to the microcapsule according to the invention. This applies in the same way to the microcapsule slurry according to the invention.

Thus, the present invention is based on the complex task of providing vegan, i.e. plant protein-based microcapsules which preferably fulfill one, several or preferably all of the following requirements:
improvement of biodegradability,
no toxic effect on humans and the environment;
maintain sufficient stability,
suitability for a wide variability with regard to the active ingredients to be encapsulated,
excellent release behavior of the encapsulated active ingredients, and
accessible by known processes for the production of microcapsules, and
components are easily accessible, i.e. can be made from bio-based or sustainably produced raw materials.

Surprisingly, it was found that this task can be solved by producing a plant protein-based microcapsule from a plant protein and a crosslinking agent in an aqueous emulsion by interfacial polymerization. Crosslinking makes it possible to form a very stable capsule shell or capsule wall that can be used to encapsulate a wide range of hydrophobic or lipophilic active ingredients. It is crucial that an internal non-aqueous phase is provided in the first step of the process according to the invention, comprising at least one aliphatic polyisocyanate as crosslinking agent and at least one hydrophobic active ingredient. The use of at least one aliphatic polyisocyanate is crucial in order to obtain a plant protein-based microcapsule which, in particular, has excellent stability with significantly improved release behavior of one or more encapsulated active ingredient(s).

### SUMMARY OF THE INVENTION

The present problem is solved by the objects of the independent patent claims.

Preferred embodiments and further embodiments result from the wording of the dependent patent claims and the following description.

In a first aspect, the invention thus relates to a process for preparing a plant protein-based microcapsule, which comprises the following steps in this order:
(i) providing an internal non-aqueous phase comprising at least one aliphatic polyisocyanate as crosslinking agent and at least one hydrophobic active ingredient and optionally at least one further crosslinking agent;
(ii) providing an external aqueous phase comprising at least one plant protein, and optionally at least one first polysaccharide and/or at least one further crosslinking agent and/or at least one polyhydroxyphenol and/or at least one protective colloid, and further optionally adjusting the pH of the aqueous phase to a pH below the isoelectric point of the plant protein;
(iii) emulsifying or dispersing the internal non-aqueous phase in the external aqueous phase, optionally in the presence of at least one stabilizer and/or at least one emulsifier, to obtain an oil-in-water emulsion or dispersion;
(iv) optionally, addition of at least one further polysaccharide and/or at least one further plant protein;
(v) first cross-linking to obtain a microcapsule slurry;
(vi) curing the microcapsule slurry at a temperature of at least 60° C. and optionally adding a further polysaccharide and/or a further plant protein;
(vii) cooling and optionally further crosslinking by addition of at least one further crosslinking agent; and
(viii) optionally, separating the microcapsules from the microcapsule slurry and, if necessary, drying the microcapsules or adjusting the viscosity of the microcapsule slurry by adding at least one thickening agent.

In a second aspect, the present invention relates to a microcapsule or a microcapsule slurry produced by the method according to the invention.

It is also an object of the present invention to provide a plant protein-based microcapsule comprising or consisting of
(a) a core comprising or consisting of at least one hydrophobic active ingredient;
(b) a capsule shell comprising or consisting of a crosslinking matrix or crosslinking units of at least one plant protein and at least one aliphatic polyisocyanate as crosslinking agent and optionally at least one polysaccharide; and optionally at least one protective colloid and/or optionally at least one further crosslinking agent.

Finally, in a further aspect, the present invention relates to the use of the plant protein-based microcapsules according to the invention or of dispersions comprising the plant protein-based microcapsules according to the invention for the manufacture of household products, textile care products, detergents, fabric softeners, cleaning agents, scent boosters, scent lotions or scent enhancers in liquid or solid form, cosmetics, personal care products, perfume compositions, agricultural products, pharmaceutical products or print coating for paper.

Furthermore, the present invention covers household products, textile care products, detergents, fabric softeners, cleaning agents, scent boosters and fragrance enhancers, cosmetics, personal care products, perfume compositions, agricultural products or pharmaceutical products comprising the plant protein-based microcapsules or corresponding slurries according to the invention.

Surprisingly, it was found that in the production of the plant protein-based microcapsules according to the invention, a combination of plant protein and subsequent crosslinking with an aliphatic polyisocyanate leads to stable plant protein-based microcapsules and thus an efficient encapsulation of lipophilic active ingredients with subsequent targeted release of these active ingredients can be ensured.

The plant protein-based and thus vegan microcapsules according to the invention thus have excellent sensory properties in addition to excellent stability. Moreover, biodegradability can be made possible due to their biobased and biodegradable building blocks. A particular advantage of the process according to the invention is that the amount of aliphatic polyisocyanate used as a crosslinking agent can be reduced compared to the prior art.

As a result, it is also possible to reduce the polyisocyanate content of the capsule wall or capsule shell material, i.e. to replace it with bio-based capsule wall components and thus increase the proportion of bio-based capsule wall components without negatively affecting the very good stability properties of the microcapsule wall.

These and other aspects, features and advantages of the present invention will become apparent to the person skilled in the art from a study of the following detailed description and claims. Any feature or variant from one aspect of the invention may be used or interchanged in another aspect of the invention. Furthermore, it is understood that the examples contained herein describe and illustrate the invention, but are not intended to limit the invention and, in particular, that the present invention is not limited to these examples.

Unless otherwise stated, all percentages are percent by weight, also referred to as wt.-%. Numerical examples given in the form “from x to y” include the stated values. Where several preferred numerical ranges are given in this format, it is understood that all ranges resulting from the combination of the different endpoints are also included.

The terms “at least one” or “not less than one” or “one or more” as used herein refer to 1 or more, for example 2, 3, 4, 5, 6, 7, 8, 9 or more.

The term “and/or” indicates that a combination exists or that an alternative is offered. The person skilled in the art can independently determine which “and” or “or” combinations appear feasible and sensible and which do not.

### FIGURES

FIG. 1 is a diagram showing a stability comparison in a fabric softener of some plant protein-based microcapsules according to the invention with already known microcapsules made of gelatin in relation to various polyisocyanates used as crosslinking agents.

FIG. 2 is a diagram showing the stability-sensory performance of microcapsules according to the invention in a fabric softener in relation to various polyisocyanates used as crosslinking agents.

FIG. 3 is a diagram showing the sensory performance of various plant protein-based microcapsules according to the invention.

FIG. 4 is a diagram showing a further comparison of the stabilities of plant-protein-based microcapsules according to the invention in a fabric softener.

FIG. 5 is a diagram showing a comparison of the stabilities of plant protein-based microcapsules according to the invention in a fabric softener in relation to the quantities of polyisocyanate used and the influence of hyaluronic acid.

FIG. 6 is a diagram showing the sensory evaluation of microcapsules according to the invention in relation to the quantities of polyisocyanate used and the influence of hyaluronic acid.

FIG. 7 is a diagram showing a further comparison of the stabilities of plant protein-based microcapsules according to the invention in relation to the amounts of polyisocyanate used and the influence of glycerol and a combination of glycerol and hyaluronic acid.

FIG. 8 is a diagram showing a sensory evaluation of microcapsules according to the invention in relation to the quantities of polyisocyanate used and the influence of hyaluronic acid and glycerol.

### DETAILED DESCRIPTION OF THE INVENTION

In a first aspect, the present invention relates to a process for preparing a plant protein-based microcapsule comprising the following steps in this order:
(i) providing an internal non-aqueous phase comprising at least one aliphatic polyisocyanate as crosslinking agent and at least one hydrophobic active ingredient and optionally at least one further crosslinking agent;
(ii) providing an external aqueous phase comprising at least one plant protein, and optionally at least one (first) polysaccharide and/or at least one further crosslinking agent and/or at least one polyhydroxyphenol and/or at least one protective colloid, and further optionally adjusting the pH of the aqueous phase to a pH below the isoelectric point of the plant protein;
(iii) emulsifying or dispersing the internal non-aqueous phase in the external aqueous phase, optionally in the presence of at least one stabilizer and/or at least one emulsifier, to obtain an oil-in-water emulsion or dispersion;
(iv) optionally, addition of at least one further polysaccharide and/or at least one further plant protein;
(v) first cross-linking while maintaining a microcapsule slurry;
(vi) curing the microcapsule slurry at a temperature of at least 60° C. and optionally adding a further polysaccharide and/or a further plant protein;
(vii) cooling and optionally further crosslinking by addition of at least one further crosslinking agent; and
(viii) optionally, separating the microcapsules from the microcapsule slurry and, if necessary, drying the microcapsules or adjusting the viscosity of the microcapsule slurry by adding at least one thickening agent.

In the context of the present invention, microcapsules are understood to be microparticles which have at least one or more active ingredients as the core material inside the capsule and are enclosed by a capsule shell or capsule wall. The active ingredients are preferably hydrophobic or lipophilic active ingredients. Such active ingredients are insoluble or poorly soluble in water, but readily soluble in fats and oils. The terms “microcapsule” and “capsule” or “hydrophobic” and “lipophilic” are used synonymously in the sense of the present invention.

In the context of the present invention, the capsule shell or capsule wall is preferably composed of several crosslinking matrices or crosslinking units, which preferably have different compositions and are generated by several process steps or process sequences, in particular crosslinking steps, during the production of the microcapsule according to the invention. The crosslinking matrix comprises at least one plant protein.

These capsule wall components are crosslinked by means of a crosslinking agent and optionally a catalyst by interfacial polymerization, optionally via specifically catalyzed mechanisms, so that a three-dimensional network comprising plant protein and aliphatic polyisocyanate as crosslinking agent and optionally at least one polysaccharide is formed.

In a first step (i) of the process according to the invention, an internal non-aqueous phase is provided which comprises at least one aliphatic polyisocyanate as crosslinking agent and at least one hydrophobic active ingredient and optionally at least one further crosslinking agent.

Polyisocyanates are R-substituted organic derivatives (R—N═C═O) of isocyanic acid (HN═C═O). Organic isocyanates are compounds in which the isocyanate group (—N═C═O) is bound to an organic radical. Polyfunctional isocyanates or polyisocyanates are compounds that contain at least two or more, i.e. 3, 4, 5, 6, 7, 8, 9, 10, 11, 12, 13, 14, 15, 20, 50, 100, 200 or even more, isocyanate groups (—N═C═O) in the molecule. Polyisocyanates with two isocyanate groups are also known as diisocyanates.

According to the invention, the at least one polyisocyanate used in step (i) is an aliphatic polyisocyanate.

It may also be provided that the at least one aliphatic polyisocyanate is linear or branched.

Surprisingly, it has been shown that the use of at least one aliphatic polyisocyanate is particularly favorable when using plant proteins and thus in the production of plant protein-based microcapsules. It is possible that the reduced reactivity of aliphatic polyisocyanates compared to aromatic polyisocyanates has a particularly advantageous effect on the stability of the plant protein-based microcapsules. In any case, the achieved stabilities of the plant protein-based microcapsules are significantly lower when aromatic polyisocyanates or mixtures with aromatic polyisocyanates are used than when aliphatic polyisocyanates are used.

In this respect, reduced reactivity with respect to the at least one polyisocyanate used as crosslinking agent is particularly preferred in the production of the plant protein-based microcapsules, i.e. in the process according to the invention. Thus, in the process according to the invention, accelerated reactivity of the polyisocyanates used and thus also the production time of the microcapsules per se is not a priority.

It is particularly preferred that at least difunctional, preferably polyfunctional polyisocyanates are used in the process according to the invention, i.e. all aliphatic and alicyclic, i.e. cycloaliphatic isocyanates are suitable, provided that they have at least one, preferably two or more reactive isocyanate groups.

Particularly preferred are aliphatic, cycloaliphatic or heterocyclic polyisocyanates, their substitution products and mixtures of the aforementioned monomeric or oligomeric compounds.

In preferred embodiments of the process according to the invention, the polyisocyanate contains an average of 2 to 5 functional —N═C═O groups. These include, for example, aliphatic or cycloaliphatic di-, tri- and higher polyisocyanates.

Among the above-mentioned polyisocyanates, diisocyanates and polyisocyanates with three functional —N—C═O groups are particularly preferred and can therefore be used primarily in the implementation of the present invention. Preferably, diisocyanates with the general structure O═C═N—R—N═C═O, where R stands for aliphatic or alicyclic radicals, can be used. Preferably, the radicals have five or more carbon atoms.

In a preferred embodiment of the process according to the invention, at least one of the aliphatic polyisocyanates or the one aliphatic polyisocyanate is a cycloaliphatic polyisocyanate with two or more isocyanate groups.

Due to the number of functional groups, an optimal cross-linking or network of the capsule wall is achieved, providing plant protein-based microcapsules with prolonged slow release of active ingredients and good stability in the consumer product.

The term “aliphatic polyisocyanate” refers to any polyisocyanate molecule which is not aromatic. In addition, the molecule comprises at least two isocyanate groups, i.e. at least 2, 3, 4, 5, 6, 7, 8, 9, 10, 11, 12, 13, 14, 15, 20, 50, 100, 200 or more isocyanate groups directly bonded to a corresponding number of different C atoms of the same aliphatic molecule, and derivatives of such compounds.

The aliphatic polyisocyanate molecule having at least two isocyanate groups, i.e. at least 2, 3, 4, 5, 6, 7, 8, 9, 10, 11, 12, 13, 14, 15, 20, 50, 100, 200 or more isocyanate groups, may further be linear, branched or cyclic and may have any substitutions including, for example, aliphatic substituents, one or more heteroatoms such as nitrogen, oxygen, phosphorus and/or sulphur, halogens such as fluorine, chlorine, bromine and/or iodine and/or other functional groups such as alkoxy groups.

The linear aliphatic polyisocyanate molecule may preferably be selected from C2- to C20- linear alkyl, preferably C3- to C15- linear alkyl, C4- to C12- linear alkyl, C5- to C10- linear alkyl, C6- to C9- linear alkyl or C7- to C8- linear alkyl. In any case, the linear aliphatic molecule does not comprise an aromatic structure.

The branched aliphatic polyisocyanate molecule may preferably be selected from C2- to C20-branched alkyl, preferably C3- to C15-branched alkyl, C4- to C12-branched alkyl, C5- to C10-branched alkyl, C6- to C9-branched alkyl, C7- to C8-branched alkyl.

For the purposes of the invention, a cycloaliphatic polyisocyanate is also to be understood and described as an aliphatic polyisocyanate.

The cyclic aliphatic polyisocyanate molecule may comprise at least 1, i.e. 1, 2, 3, 4 or more non-aromatic ring structures, the ring structure itself preferably consisting only of C atoms. Of course, the C atoms of the ring structure can carry suitable substituents. The at least 1 ring structures preferably consist independently of one another of 3, 4, 5, 6, 7 or 8-membered rings. Preferably, the cyclic aliphatic molecule comprises 2 to 20 C atoms, such as 3 to 15 C atoms, 4 to 12 C atoms, 5 to 10 C atoms, 6 to 9 C atoms or 7 to 8 C atoms.

The linear, branched or cyclic aliphatic polyisocyanate can be present as a monomer or polymer. A monomeric polyisocyanate is a molecule that is not linked to another molecule, in particular not by one or more crosslinking agents. A polymeric polyisocyanate comprises at least two monomers which are linked to one another by one or more crosslinking agents. The at least two monomers do not necessarily have to be the same monomers, but may also be different. A polymeric polyisocyanate preferably comprises at least 2 or more monomers, i.e. at least 2, 3, 4, 5, 10, 20, 30, 40, 50, 100 or more monomers linked together by at least one crosslinking agent.

The linear, branched or cyclic aliphatic polyisocyanate preferably has a limited size/molecular weight which allows reactivity with the one or more crosslinking agents. Examples of suitable molecular weights preferably include approx. 100 g/mol to 5·10⁴; g/mol, preferably 120 g/mol to 2·10⁴ g/mol, 140 g/mol to 10⁴; g/mol 160 g/mol to 5·10³ g/mol, 180 g/mol to 2·10³ g/mol, 200 g/mol to 10³ g/mol, 220 g/mol to 900 g/mol, 240 g/mol to 800 g/mol, 260 g/mol to 700 g/mol, 280 g/mol to 600 g/mol, 300 g/mol to 500 g/mol, 320 g/mol to 450 g/mol or 340 g/mol to 400 g/mol.

Any number of different linear, branched and/or cyclic aliphatic polyisocyanates can be used within the scope of the invention. For example, at least one or more, i.e. at least 1, 2, 3, 4, 5, 6, 7, 8, 9 or 10 different linear aliphatic polyisocyanates may be used. For example, at least one or more, i.e. at least 1, 2, 3, 4, 5, 6, 7, 8, 9 or 10 different branched aliphatic polyisocyanates may also be used. For example, at least one or more, i.e. at least 1, 2, 3, 4, 5, 6, 7, 8, 9 or 10 different branched cyclic polyisocyanates can be used as crosslinking agents.

Preferably, derivatives of linear, branched and/or cyclic aliphatic polyisocyanates are used. A derivative, as used herein, is understood in its broadest sense as a compound derived from a compound by a chemical reaction. Examples of derivatives include oligomers and/or adducts of the above-mentioned linear or branched aliphatic polyisocyanates. Preferred oligomers are biurets, isocyanurates, uretdiones, iminooxadiazinediones and preferred adducts are trimethylolpropane adducts. These oligomers/adducts are well known in the prior art and are disclosed, for example, in U.S. Pat. Nos. 4,855,490 A or 4,144,268 A.

Preferably, the aliphatic polyisocyanate is only present in monomeric form and/or dimerized form (as isocyanate) or in oligomeric form.

The derivatives of the linear, branched or cyclic polyisocyanates and/or mixtures thereof can also be obtained by reacting the polyisocyanates with polyalcohols (e.g. glycerol), polyamines, polythiols (e.g. dimercaprol).

The isocyanate compounds as defined above expressly include the various isomers, if any, alone or in combination. For example, methylene bis(cyclohexyl isocyanate) (H12MDI) comprises 4,4′-methylene bis(cyclohexyl isocyanate), 2,4′-methylene bis(cyclohexyl isocyanate) and/or 2,2′-methylene bis(cyclohexyl isocyanate).

Exemplary aliphatic polyisocyanates include those that are commercially available, e.g. BAYHYDUR N304 and BAYHYDUR N3Q5, which are aliphatic water-dispersible polyisocyanates based on hexamethylene diisocyanate, DESMODUR N3400, DESMODUR N3600, DESMODUR N3700 and DESMODUR N3900, which are low-viscosity polyfunctional aliphatic polyisocyanates based on hexamethylene diisocyanate, polyfunctional aliphatic polyisocyanates based on hexamethylene diisocyanate, and DESMODUR 3600 and DESMODUR N100, which are aliphatic polyisocyanates based on hexamethylene diisocyanate, each of which is available from Bayer Corporation, Pittsburgh, PA.

According to another preferred variant of the present invention, the linear or branched aliphatic and/or cycloaliphatic polyisocyanates is or are selected from the group consisting of pentamethylene diisocyanate (PDI, such as Stabio D-370N or D-376N from Mitsui Chemicals Inc, Japan), hexamethylene diisocyanate (HDI), ethyl ester lysine triisocyanate, lysine diisocyanate ethyl ester and derivatives thereof, preferably wherein each of said derivatives comprises more than one isocyanate group and optionally further comprises one or more groups selected from the group consisting of biuret, isocyanurate, uretdione, iminooxadiazinedione and trimethylolpropane adduct and/or wherein the cyclic aliphatic polyisocyanate(s) is/are selected from the group consisting of isophorone diisocyanate (IPDI), 1,3-bis(isocyanatomethyl) cyclohexane (H6XDI, such as Takenate) or is/are selected 600 from Mitsui Chemicals Inc., Japan), 1,2-bis(isocyanatomethyl) cyclohexane, 1,4-bis(isocyanato-methyl) cyclohexane, methylenebis (cyclohexyl isocyanate)) (H12MDI) and derivatives thereof, preferably wherein each of said derivatives comprises more than one isocyanate group and optionally further comprises one or more groups selected from the group consisting of biuret, isocyanurate, uretdione, iminooxadiazinione and trimethylolpropane adduct (such as TMP adduct) of H6XDI, in particular Takenate D-120N from Mitsui Chemicals Inc, Japan).

Aliphatic polyisocyanates obtained from renewable raw materials such as PDI (Stabio D-370N or D-376N from Mitsui Chemicals Inc., Japan) are particularly preferred. It was found that such aliphatic polyisocyanates obtained from renewable raw materials do not negatively affect the quality/properties of the core-shell capsules.

In a variant of the process according to the invention, the polyisocyanate used in the preparation of the plant protein-based microcapsules according to the present invention is used as the sole polyisocyanate component, i.e. without the admixture of another polyisocyanate component different therefrom.

Examples of the monomeric polyisocyanates which can be used according to the invention and which contain at least two polyisocyanate groups are
Ethylene diisocyanate, trimethylene diisocyanate, 1,4-tetramethylene diisocyanate, 1,6-hexamethyldiisocyanate, ethylene diisothiocyanate, tetramethylene diisothiocyanate, hexamethylene diisothiocyanate, cyclobutane-1,3-diisocyanate, cyclohexane-1,3-diisocyanate, cyclohexane-1,4-diisocyanate or mixtures of the aforementioned compounds.

As polymerization-capable compounds containing at least two polyisocyanate groups, industrially produced di- and poly-isocyanates are preferred, for example HDI: hexamethylene diisocyanate-(1,6) and/or IPDI: isophorone diisocyanate.

Other particularly preferred monomeric polyisocyanate compounds are: Diisocyanates such as 1,4-diisocyanatobutane, 1,6-diisocyanatohexane, 1,5-diisocyanato-2,2-dimethylpentane, 2,2,4- and 2,4,4-trimethyl-1,6-diisocyanatohexane, 1,10-diiso-cyanatodecane, 1,3- and 1,4-diisocyanatocyclohexane, 1-polyisocyanato-3,3,5-trimethyl-5-polyisocyanatomethylcyclohexane (isophorone diisocyanate), 4,4′-diisocyanato-dicyclohexylmethane, 2,4- and 2,6-diisocyanatomethylcyclohexane and mixtures thereof.

Other specific examples of diisocyanates include, for example, 1-methyl-2,4-diisocyanatocyclohexane, 1,6-diisocyanato-2,2,4-trimethylhexane, 1,6-diiso-cyanato-2,4,4-trimethylhexane, 1-isocyanatomethyl-3-isocyanato-1,5,5-trimethyl-cyclo-hexane, chlorinated and brominated diisocyanates, phosphorus-containing diisocyanates, tetra-methoxybutane-1,4-diisocyanate, butane-1,4-diisocyanate, (HDI), dicyclohexylmethane diisocyanate, cyclohexane-1,4-diisocyanate, ethylene diisocyanate.

In particular, the use of longer-chain aliphatic diisocyanates with six, seven, eight, nine, ten or even more carbon atoms can lead to the formation of more stable capsule shells or capsule walls.

In a particularly preferred embodiment, the internal non-aqueous phase comprises a mixture of two or more different polymerizable polyisocyanates, for example polyisocyanates with different chain lengths, which can form mixed polymers.

The process according to the invention may provide for the use of a combination of at least two different aliphatic polyisocyanates.

In such a combination, the different reaction rates of the polyisocyanates are exploited. Short-chain aliphatic polyisocyanates, i.e. aliphatic polyisocyanates with one to five carbon atoms, preferably three to five carbon atoms, can enable higher reaction rates compared to longer-chain analogs.

In a further preferred further development of the invention, the different aliphatic polyisocyanates therefore also have different chain lengths. In this context, longer-chain polyisocyanates preferably have six, seven, eight, nine, ten, eleven, twelve, thirteen, fourteen, 20, 25 or more carbon atoms, but even more preferably they have six to twelve carbon atoms and particularly preferably six to eight carbon atoms. By shorter-chain polyisocyanates are meant polyisocyanates with one to five carbon atoms and preferably polyisocyanates with three to five carbon atoms.

It is possible to use a combination of a short-chain aliphatic polyisocyanate (C1, C2, C3, C4, C5) and a long-chain aliphatic polyisocyanate (C6, C7, C8, C9, C10, C11, C12, C13, C14, C15, C20, C25 or more).

Particularly preferred in this context is the use of a mixture of different aliphatic polyisocyanates with two or more isocyanate groups with chain lengths of one to twelve carbon atoms in the chain, preferably three to eight carbon atoms and particularly preferably four to seven carbon atoms, for the production of the plant-protein-based microcapsules according to the invention.

Aliphatic polyisocyanates are particularly preferable in this context, not least because of their chemical relationship to biobased systems. For example, both lysine and 1,5-diiso-cyanato-pentane show the same degradation product, 1,5-diaminopentane, and are therefore particularly suitable for improved biodegradability of microcapsules, taking into account environmental aspects.

Primary embodiments comprise mixtures of longer-chain and shorter-chain diisocyanates in any desired mixing ratios. Preferably, the mixing ratio of longer-chain diisocyanates to shorter-chain diisocyanates is in a range from 4 to 1 to 1 to 4 and particularly preferably from 2 to 1 to 1 to 2.

Preferably, the process according to the invention can provide that in the previously described combination of a short-chain aliphatic polyisocyanate and a long-chain aliphatic polyisocyanate, the polyisocyanates are present in a mixture of monomeric or oligomeric or polymeric form.

Preferably, this results in the following combinations for use in the method according to the invention:
short-chain aliphatic polyisocyanate (monomer or oligomer or polymer) and short-chain aliphatic polyisocyanate (monomer or oligomer or polymer);
short-chain aliphatic polyisocyanate (monomer or oligomer or polymer) and long-chain aliphatic polyisocyanate (monomer or oligomer or polymer);
long-chain aliphatic polyisocyanate (monomer or oligomer or polymer) and short-chain aliphatic polyisocyanate (monomer or oligomer or polymer);
long-chain aliphatic polyisocyanate (monomer or oligomer or polymer) and long-chain aliphatic polyisocyanate (monomer or oligomer or polymer);
with the previously described definitions for short-chain and long-chain.

The choice of at least two aliphatic polyisocyanates with different chain lengths and different degrees of polymerization can lead to a significant gain in stability and performance (fragrance release in the case of fragrance or fragrance capsules) due to the different reaction rates of the polyisocyanate components, dissociations and cross-linking structures.

The above-mentioned polyisocyanate combinations or polyisocyanate mixtures of two different aliphatic polyisocyanates can be used to produce particularly stable and better, i.e. more densely branched, cross-links within the capsule shell.

Thus, the method described herein can be used to produce high-performance (fragrance or odorant release) plant protein-based microcapsules, which are made from a mixture of two different aliphatic polyisocyanates. Such plant protein-based microcapsules are very stable and are characterized by outstanding fragrance storage properties, which in turn is reflected in a better performance (fragrance or odorant release) of the capsules, for example in the field of fragrance or odorant encapsulation.

The use of two different aliphatic polyisocyanates can result in microcapsules that even exceed the stability of microcapsules from single polyisocyanate systems.

The microcapsule made from an aliphatic-aliphatic polyisocyanate mixture is significantly improved compared to a microcapsule made from an aliphatic-aromatic polyisocyanate mixture, at least in terms of stability, as is illustrated in the following embodiment examples. Accordingly, the present invention basically provides for the use of aliphatic polyisocyanates in the combinations described above. The use of any aromatic polyisocyanates is in any case explicitly excluded in the present invention.

The proportion of the crosslinking agent(s), preferably the polyisocyanate(s), in the internal non-aqueous phase is in a range from 0.1 to 5% by weight, preferably in a range from 0.2 to 4% by weight, based on the total weight of the non-aqueous phase. Most preferably, the crosslinking agent is used in the internal non-aqueous phase in a range of 0.5 to 2% by weight, based on the total weight of the non-aqueous phase. The total weight of the non-aqueous phase is composed of all components of the non-aqueous phase.

The crosslinking agent is added to the internal non-aqueous phase either as such, for example as a solid, or in the form of an aqueous solution.

In order to obtain plant protein-based microcapsules which have a very good balance between stability and sensory properties (fragrance or odorant release) and also have the potential for biodegradability, a particularly advantageous embodiment of the process according to the invention may provide that the total amount of polyisocyanate, based on the amount of wall-forming agent, is from 15 to 70% by weight, preferably from 20 to 50% by weight, particularly preferably from 25 to 35% by weight, or wherein the total amount of the polyisocyanates used as crosslinking agents together is 0.5% to 4%, preferably 1% to 3%, particularly preferably 1.5% to 2.5% based on the total amount of hydrophobic active ingredient used, preferably fragrance or odorant, particularly in the core of the capsule.

Wall formers within the meaning of the invention are all solid components of the internal and external phases as well as any optionally added polysaccharides, in particular further crosslinking agents and/or polyhydroxyphenols and/or catalysts and, in the present disclosure, all other suitable substances.

In a further advantageous further development of the process according to the invention, it may be provided that in step (i) a non-internal non-aqueous phase is provided comprising an aliphatic polyisocyanate as crosslinking agent and a cycloaliphatic polyisocyanate as crosslinking agent, wherein the aliphatic polyisocyanate and the cycloaliphatic polyisocyanate are used in a respective molar ratio of 85:15 to 15:85.

Surprisingly, plant protein-based microcapsules can be produced in this way, which have particularly good stability properties combined with very good sensory properties (fragrance or odorant release). In addition, the total content of polyisocyanates used as crosslinking agents can be reduced, which has a positive impact on the environment.

In a further advantageous further development of the process according to the invention, it may be provided that in step (i) a non-internal non-aqueous phase is provided comprising two different aliphatic polyisocyanates as crosslinking agents, the two aliphatic polyisocyanates being used in a respective molar ratio of 85:15 to 15:85.

With regard to the two aliphatic polyisocyanates, reference is made to the detailed explanations in the overall context of the disclosure, which are equally applicable with regard to the stated molar ratios, where appropriate.

Surprisingly, it has been found that when the two aliphatic polyisocyanates are used in a respective molar ratio of 85:15 to 15:85, the plant protein-based microcapsules produced in this way each exhibit very good stability properties and very good sensory properties (fragrance or odor release).

In order to obtain plant protein-based microcapsules which are optimized with respect to both their stability behavior and their sensory properties, it may be provided in a particularly preferred aspect of the process according to the invention that in step (i) an internal non-aqueous phase comprising three crosslinking agents is provided, wherein the three crosslinking agents are aliphatic or cycloaliphatic polyisocyanates different from each other, wherein preferably at least one cycloaliphatic polyisocyanate and at least one aliphatic polyisocyanate is present, wherein the three polyisocyanates are each used together in amounts of from 20% to 60% based on the total weight of the three polyisocyanates.

Preferably, three cylcloaliphatic polyisocyanates can be used.

In this context, it can also be particularly advantageous to use a cycloaliphatic polyisocyanate and two aliphatic polyisocyanates that are different from one another.

With regard to the three polyisocyanates, reference is made to the detailed explanations in the overall context of the disclosure, which are equally applicable with regard to the stated proportions, where appropriate.

In a moreover advantageous further development of the process according to the invention, it may be provided that the three cycloaliphatic polyisocyanates are each used in equal proportions.

Furthermore, it may be provided that the cycloaliphatic polyisocyanate and the two aliphatic polyisocyanates, which are different from each other, are each used in equal proportions.

In this way, optimized polyisocyanate mixtures can be provided, which have a particularly advantageous effect on the stability of the microcapsules obtained and their sensory properties (odor or fragrance release), whereby it has surprisingly been found that the total amount of polyisocyanates used can be reduced.

In addition, the advantages or effects of the polyisocyanates described in the overall disclosure apply additionally and/or supplementarily to the molar ratios and/or quantitative ratios described herein, if appropriate.

To improve the crosslinking of the at least one polysaccharide and/or the at least one plant protein, at least one further crosslinking agent can be added to the internal non-aqueous phase. In one embodiment, the further crosslinking agent is different from the (first) crosslinking agent. In another embodiment, the at least one further crosslinking agent may be the same as the crosslinking agent. It is also possible that the crosslinking agent comprises the at least one further crosslinking agent.

It may be provided that in step (i) at least one further crosslinking agent and/or in step (ii) and/or in step (vii) a further crosslinking agent is added.

The at least one further crosslinking agent in the case of addition in step (i) or in the case of addition in step (ii) and/or in step (vii) is/are selected from the group consisting of transglutaminase, peroxidase, secondary plant substances selected from the group consisting of polyphenols, polyhydroxyphenols, in particular tannin, gallic acid, ferulic acid, hesperidin, cinnamaldehyde, vanillin, carvacrol and mixtures of two or more of the aforementioned crosslinking agents. In this respect, the at least one further crosslinking agent can be a polyphenol and/or a polyhydroxyphenol.

Transglutaminase is an enzyme that catalyzes cross-linking via isopeptide bonds between two amino acids, glutamine and lysine. The phenolic groups of the secondary plant substances cross-link the peptides via hydrogen bonds. The aldehydes, cinnamaldehyde and vanillin, react covalently with the free amino groups of the proteins via the reactive aldehyde groups.

Cinnamaldehyde, tannin, ferulic acid and gallic acid are particularly preferred among the other crosslinking agents mentioned above.

Tannin is particularly preferred. The sensory properties of the microcapsules according to the invention can advantageously be significantly improved.

Very preferably, it may be provided that tannin is added in step (i) as the at least one further crosslinking agent and/or in step (ii) and/or in step (vii) as the one further crosslinking agent.

Particularly advantageous combinations of crosslinking agent and further crosslinking agent are:
polyisocyanate plus transglutaminase;
polyisocyanate plus peroxidase;
polyisocyanate plus polyphenol;
polyisocyanate plus tannin;
polyisocyanate plus gallic acid;
polyisocyanate plus ferulic acid;
polyisocyanate plus hesperidin;
polyisocyanate plus cinnamaldehyde;
polyisocyanate plus vanillin;
polyisocyanate plus carvacrol; or
polyisocyanate plus a mixture of two or more of the other crosslinking agents described above.

The further crosslinking agent can be added to the internal non-aqueous phase either as such, for example as a solid, or in the form of a solution.

The combined use of at least one crosslinking agent and at least one further crosslinking agent, which are different from each other, results in microcapsules with significantly improved stability and thus in a lower proportion of hydrophobic active ingredient leaking out.

In step (i) of the process according to the invention, the at least one crosslinking agent is first substantially dissolved together with the at least one or more active ingredient(s) to be encapsulated, if appropriate in an inert, non-aqueous solvent or a solvent mixture of inert non-aqueous solvents. The term “substantially dissolved” is understood to mean that at least 90 wt.-%, preferably at least 98 wt.-%, even more preferably 99.9 wt.-% of the aforementioned component are dissolved in the solvent or in the solvent mixture in order to be able to use them in the present process. Preferably, the at least one polyisocyanate and the at least one active ingredient to be encapsulated are completely dissolved in the solvent or in the solvent mixture. If a solvent does not ensure sufficient solubility of the isocyanates, it is possible to overcome this disadvantage by using suitable solubility promoters.

Preferred solvents for the internal non-aqueous phase are immiscible with water and do not react with the isocyanate component(s) or the active ingredient component(s) and have little or no odor in the amounts used.

The term “solvent” in the context of the present invention includes all types of oil bodies or oil components, in particular vegetable oils such as rapeseed oil, sunflower oil, soybean oil, olive oil and the like, modified vegetable oils, e.g. alkoxylated sunflower or soybean oil, synthetic (tri)glycerides such as e.g. technical mixtures of mono-, di- and triglycerides of C6 to C22 fatty acids, fatty acid alkyl esters, e.g. methyl or ethyl esters of vegetable oils (Agnique® ME 18 RD-F, Agnique® ME 18 SD-F, Agnique® ME 12C-F, Agnique® ME1270), fatty acid alkyl esters based on these C6 to C22 fatty acids, mineral oils and mixtures thereof. Examples of suitable and preferred lipophilic solvents are Guerbet alcohols based on fatty alcohols with 6 to 18, preferably 8 to 10, carbon atoms, esters of linear C6 to C22 fatty acids with linear or branched C6 to C22 fatty alcohols or -esters of branched C6 to C13 carboxylic acids with linear or branched C6 to C22 fatty alcohols, such as myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl stearate, stearyl erucate, isostearyl myristate, isostearyl palmitate, isostearyl stearate, isostearyl isostearate, isostearyl oleate, isostearyl behenate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate.

Also suitable are esters of linear C6 to C22 fatty acids with branched alcohols, in particular 2-ethylhexanol, esters of C18 to C38 alkyl hydroxycarboxylic acids with linear or branched C6 to C22 fatty acids, in particular dioctylalate, esters of linear or branched fatty acids with polyhydric alcohols (such as propylene glycol, dimer diol or trimer triol) and/or guerbet alcohols, triglycerides based on C6 to C10 fatty acids, liquid mono-/di-/triglyceride mixtures of C6 to C18 fatty acids, esters of C6 to C22 fatty alcohols and/or Guerbet alcohols with aromatic carboxylic acids, in particular benzoic acid, esters of C2 to C12 dicarboxylic acids with linear or branched alcohols with 1 to 22 carbon atoms or polyols with 2 to 10 carbon atoms and 2 to 6 hydroxyl groups, vegetable oils, branched primary alcohols, substituted cyclohexanes, linear or branched C6 to C22 fatty alcohol carbonates, such as dicaprylyl carbonate (Cetiol® CC); Guerbet carbonates based on fatty alcohols with 6 to 18, preferably 8 to 10 carbon atoms, benzoic acid esters with linear or branched C6 to C22 alcohols, linear or branched, symmetrical or asymmetrical dialkyl ethers with 6 to 22 carbon atoms per alkyl group, such as dicaprylyl ether, ring-opening products of epoxidized fatty acid esters with polyols, silicone oils (cyclomethicones, silicone methicone grades, etc.), aliphatic or naphthenic hydrocarbons such as squalane, squalene or dialkylcyclohexanes and/or mineral oils.

Preferred solvents are in particular also esters of linear C6 to C22 fatty acids with branched alcohols, esters of C18 to C38 alkyl hydroxycarboxylic acids with linear or branched C6 to C22 fatty alcohols, linear or branched C6 to C22 fatty alcohols, in particular dioctyl malates, esters of linear or branched fatty acids with polyhydric alcohols, such as, for example propylene glycol, dimer diol or trimertriol, and/or Guerbet alcohols, triglycerides based on C6 to C10 fatty acids, liquid mono/di/triglyceride mixtures based on C6 to C18 fatty acids, esters of C6 to C22 fatty alcohols and/or Guerbet alcohols with aromatic carboxylic acids, in particular benzoic acid, esters of C2 to C12 dicarboxylic acids with linear or branched alcohols with 1 to 22 carbon atoms or polyols with 2 to 10 carbon atoms and 2 to 6 hydroxyl groups, vegetable oils, branched primary alcohols, substituted cyclohexanes, linear and branched C6 to C22 fatty alcohol carbonates, such as e.g. dicaprylyl carbonates (Cetiol™ CC), Guerbet carbonates based on fatty alcohols with 6 to 18, preferably 8 to 10, carbon atoms, esters of benzoic acid with linear or branched C6 to C22 alcohols, linear or branched, symmetrical or asymmetrical dialkyl ethers with 6 to 22 carbon atoms per alkyl group, e.g. dicaprylyl ether (Cetiol™ OE), ring-opening products of epoxidized fatty acid esters with polyols, silicone oils (cyclomethicones, silicon methicone types etc.) and/or aliphatic or naphthenic hydrocarbons, e.g. squalane, squalene or dialkylcyclohexanes.

Furthermore, liquid linear and/or branched and/or saturated or unsaturated hydrocarbons or any desired mixtures thereof can be used as solvents in the context of the present invention. These can be, for example, alkanes with 4 to 22, preferably 6 to 18 carbon atoms, or any desired mixtures thereof.

Particularly suitable inert solvents for the internal non-aqueous phase are alkylaromatic hydrocarbons such as diisopropylnaphthalene or substituted biphenyls, chlorinated diphenyl, paraffins, chlorinated paraffin, natural vegetable oils such as cottonseed oil, peanut oil, palm oil, tricresyl phosphate, silicone oil, dialkyl phthalates, dialkyl adipates, partially hydrogenated terphenyl, alkylated biphenyl, alkylated naphthalene, diaryl ether, aryl alkyl ether and higher alkylated benzene, benzyl benzoate, isopropyl myristate as well as any mixtures of these hydrophobic solvents and mixtures of one or more of these hydrophobic solvents with kerosene, paraffins and/or isoparaffins.

Preferably, vegetable oils triglycerides, benzyl benzoate or isopropyl myristate are used as solvents for providing the internal non-aqueous phase. Most preferred are vegetable oils selected from the group consisting of palm oil, soybean oil, rapeseed oil, sunflower oil, palm kernel oil, cottonseed oil, peanut oil, corn oil, coconut oil, olive oil, sesame oil, linseed oil, safflower oil, modified vegetable oils and mixtures thereof.

The above-mentioned solvents are used in the process according to the invention either individually or as a mixture of two or more solvents.

In an alternative and preferred variant of the process according to the invention, the at least one polyisocyanate is dissolved directly in a solution of the at least one active ingredient, preferably one or more fragrance or flavoring agent(s) or a perfume oil, so that essentially no solvent, as described above, is present in the core of the plant protein-based microcapsule according to the invention. The avoidance of a solvent in the microcapsule core is advantageous in that it reduces manufacturing costs and takes environmental aspects into account.

The fragrances or flavorings are particularly dissolved in solvents that are commonly used in the perfume or flavoring industry. The solvent is preferably not an alcohol, since alcohols react with the isocyanates. Examples of suitable solvents are diethyl phthaloate, isopropyl myristate, Abalyn® (rosin resins, available from Eastman), benzyl benzoate, ethyl citrate, limonene or other terpenes or isoparaffins. Preferably, the solvent is highly hydrophobic. Preferably, the fragrance or flavoring solution comprises less than 30% solvent. More preferably, the fragrance or flavoring solution comprises less than 20% and even more preferably less than 10% solvent, all of these percentages being defined by weight relative to the total weight of the fragrance or flavoring solution. Most preferably, the fragrance or flavorant is substantially free of solvent.

If the at least one hydrophobic active ingredient is already present in a mixture with a solvent or solvent mixture, the use of an inert solvent or solvent mixture is not necessary. In such a case, the at least one crosslinking agent can be mixed directly with the hydrophobic active ingredient to obtain an internal non-aqueous phase.

In principle, any material suitable for inclusion in microcapsules can be used as the active ingredient to be encapsulated or as the core material for producing the microcapsules according to the invention. The active substances to be encapsulated are preferably hydrophobic, i.e. water-insoluble or water-immiscible liquids or solids as well as suspensions. These are predominantly non-polar substances. Such hydrophobic substances are almost always lipophilic, i.e. they dissolve well in fat and oil.

In the context of the present description, the core material is a hydrophobic active ingredient, i.e. a substance that has a specific effect or causes a specific reaction, for example a medicinal substance, a plant protection agent, a cosmetic active ingredient, a food active ingredient, or the like.

The at least one active ingredient to be encapsulated, which is used in the method according to the invention, is a hydrophobic or lipophilic active ingredient. This ensures that the active ingredient to be encapsulated is in the internal non-aqueous phase during the production of the microcapsule according to the invention and does not mix with the external aqueous phase, as otherwise no emulsion can form and no deposition of the capsule wall material on the droplet surface can take place. As a result, the lipophilic active ingredient is completely trapped inside the microcapsule as core material during the subsequent emulsification and cross-linking of the capsule wall components. The internal non-aqueous phase formed in this way is characterized by its organic hydrophobic, oily character.

As described above, it may be provided that a further crosslinking agent is added in step (ii). This may be a crosslinking agent selected from the group consisting of transglutaminase, peroxidase, secondary plant substances selected from the group consisting of polyphenols, polyhydroxyphenols, in particular tannin, gallic acid, ferulic acid, hesperidin, cinnamaldehyde, vanillin, carvacrol and mixtures of two or more of the above-mentioned crosslinking agents.

It is possible that the amount of the further crosslinking agent which is optionally added in step (ii) is 0.1% to 1.0%, preferably 0.15% to 0.5%, particularly preferably 0.17% to 0.23% relative to the total amount of the external aqueous phase.

In an advantageous embodiment of the present invention, the at least one lipophilic or hydrophobic active ingredient is in particular a lipophilic or hydrophobic fragrance or flavoring or a lipophilic or hydrophobic perfume oil or flavor (fragrance or flavoring mixture), a cooling agent, a TRPV1 or a TRPV3 modulator, a substance that causes a sharp taste or a warm or hot sensation on the skin or mucous membranes or a fizzing or tingling sensation in the mouth or throat, or active ingredients with an astringent effect, a pesticide, a biocide, an insecticide, a substance from the group of repellents, a food additive, a cosmetic active ingredient, a pharmaceutical active ingredient, a dye, a dye precursor, an agrochemical, a dye, a luminous paint, an optical brightener, a solvent, a wax, a silicone oil, a lubricant, a printing coating for paper, or a mixture of two or more of the aforementioned active ingredients.

In a preferred variant of the present invention, hydrophobic or lipophilic active ingredients are in particular hydrophobic fragrance or odiferous substances or mixtures of two or more fragrances or odiferous substances (perfume oils) or hydrophobic flavoring substances or flavoring substance mixtures of two or more flavoring substances (flavors) or also biogenic principles.

In a preferred embodiment according to the first and/or second aspect of the present invention, the microcapsules comprise a core material in the form of a hydrophobic single fragrance or single odorant, wherein the core material comprises at least one single fragrance or single odorant or mixtures thereof, selected from one or more of the following groups: extracts of natural raw materials and also fractions thereof or components isolated therefrom; single fragrances from a group of hydrocarbons; aliphatic alcohols; aliphatic aldehydes and acetals; aliphatic ketones and oximes; aliphatic sulfur-containing compounds; aliphatic nitriles; esters of aliphatic carboxylic acids; formates, acetates, propionates, isobutyrates, butyrates, isovalerates, pentanoates, hexanoates, crotonates, tiglinates and 3-methyl-2-butenoates of acyclic terpene alcohols; acyclic terpene aldehydes and ketones and their dimethyl and diethyl acetals; formates, acetates, propionates, isobutyrates, butyrates, isovalerates, pentanoates, hexanoates, crotonates, tiglinates and 3-methyl-2-butenoates of cyclic terpene alcohols; cyclic terpene aldehydes and ketones; cyclic alcohols; cyclic and cycloaliphatic ethers; cyclic and macrocyclic ketones; cycloaliphatic aldehydes; cycloaliphatic ketones; esters of cyclic alcohols; esters of cycloaliphatic alcohols; esters of cycloaliphatic carboxylic acids; aromatic hydrocarbons; araliphatic alcohols; esters of araliphatic alcohols and aliphatic carboxylic acids; araliphatic ethers; aromatic and araliphatic aldehydes; aromatic and araliphatic ketones; aromatic and araliphatic carboxylic acids and their esters; nitrogen-containing aromatic compounds; phenyl ethers and phenyl esters; heterocyclic compounds; lactones; and mixtures of the aforementioned active ingredients.

Suitable fragrances and flavors for the preparation of the capsules according to the invention are described, for example, in “Fragrances”, in Steffen Arctander, in “Perfume and Flavor Chemicals”, self-published, Montclair, N.J. 1969; H. Surburg, J. Panten, in “Common Fragrance and Flavor Materials”, 5th edition, Wiley-VCH, Weinheim 2006.

Preferably, the plant protein-based microcapsules according to the invention comprise a core material in the form of a hydrophobic single fragrance or single flavoring agent, wherein the core material comprises at least one single fragrance or single flavoring agent selected from one or more of the following groups:
hydrocarbons, such as 3-carene; a-pinene; beta-pinene; alpha-terpinene; gamma-terpinene; p-cymene; bisabolene; camphene; caryophyllene; cedrene; farnesene; limonene; longifolene; myrcene; ocimene; valencene; (E,Z)-1,3,5-undecatriene;
aliphatic alcohols, such as hexanol; octanol; 3-octanol; 2,6-dimethylheptanol; 2-methylheptanol, 2-methyloctanol; (E)-2-hexenol; (E)- and (Z)-3-hexenol; 1-octen-3-ol; Mixture of 3,4,5,6,6-pentamethyl-3,4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
aliphatic aldehydes and their acetals, such as hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-5,9-undecadienal; heptanaldiethyl acetal; 1,1-dimethoxy-2,2,5-trimethyl-4-hexene; citronellyloxyacetaldehyde;
aliphatic ketones and their oximes, such as 2-heptanone; 2-octanone; 3-octanone; 2-nonanone; 5-methyl-3-heptanone; 5-methyl-3-heptanone oxime; 2,4,4,7-tetramethyl-6-octen-3-one;
aliphatic sulphur-containing compounds, such as 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;
aliphatic nitriles, such as 2-nonenonitrile; 2-tridecenonitrile; 2,12-tridecenonitrile; 3,7-dimethyl-2,6-octadienonitrile; 3,7-dimethyl-6-octenonitrile;
aliphatic carboxylic acids and their esters, such as (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octene-3-acetate; ethyl butyrate; butyl butyrate; isoamyl butyrate; hexyl butyrate; (E)- and (Z)-3-hexenyl isobutyrate; hexyl crotonate; ethyl isovalerate; ethyl 2-methyl pentanoate; ethyl hexanoate; allyl hexanoate; ethyl heptanoate; allyl heptanoate; ethyl octanoate; ethyl (E,Z)-2,4-decadienoate; methyl 2-octinate; methyl 2-noninate; allyl 2-isoamyloxyacetate; methyl 3,7-dimethyl-2,6-octadienoate;
acyclic terpene alcohols, such as citronellol; geraniol; nerol; linalool; lavadulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatriene-1-ol; and their formates, acetates, propionates, isobutyrates, butyrates, isovalerates, pentanoates, hexanoates, crotonates, tiglinates, 3-methyl-2-butenoates;
acyclic terpene aldehydes and ketones, such as geranial; neral; citronellal; 7-hydroxy-3,7-dimethyloctanal; 7-methoxy-3,7-dimethyloctanal; 2,6,10-trimethyl-9-undecenal; geranylacetone; and the dimethyl and diethyl acetals of geranial, neral, 7-hydroxy-3,7-dimethyloctanal;
cyclic terpene alcohols, such as menthol; isopulegol; a-terpineol; terpinenol-4; menthane-8-ol; menthane-1-ol; menthane-7-ol; borneol; isoborneol; linalool oxide; nopol; cedrol; ambrinol; vetiverol; guaiol; and their formates, acetates, propionates, isobutyrates, butyrates, isovalerianates, pentanoates, hexanoates, crotonates, tiglinates, 3-methyl-2-butenoates;
cyclic terpene aldehydes and ketones, such as menthone; isomenthone; 8-mercaptomenthan-3-one; carvone; camphor; fenchone; α-ionone; beta-ionone; α-n-methylionone; beta-n-methylionone; α-isomethylionone; beta-isomethylionone; α-irone; ß-irone; α-damascenone; beta-damascenone; gamma-damascenone; d-damascenone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalen-8(5H)-one; nootkatone; dihydronootkatone; α-sinensal; beta-sinensal; acetylated cedarwood oil (methylcedrylketone);
cyclic alcohols, such as 4-tert-butylcyclohexanol; 3,3,5-trimethylcyclohexanol; 3-isocamphylcyclohexanol; 2,6,9-trimethyl-(Z2,Z5,E9)-cyclododecatrien-1-ol; 2-isobutyl-4-methyltetrahydro-2H-pyran-4-ol; from the group of cycloaliphatic alcohols, such as 3,3,3-trimethyl-cyclohexylmethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;
cyclic and cycloaliphatic ethers, such as cineole; cedryl methyl ether; cyclododecyl methyl ether; (ethoxymethoxy)cyclododecane; a-cedrene epoxide; 3a,6,6,9a-tetramethyldodecahydronaphtho[2,1-b]furan; 3a-ethyl-6,6,9a-trimethyl-dodecahydronaph-tho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methylpropyl)-1,3-dioxane;
cyclic ketones, such as 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; 5-cyclohexadecen-1-one; 6,7-dihydro-1,1,2,3,3-pentamethyl-4(5H)-indanone; 9-cycloheptadecen-1-one; cyclopentadecanone; cyclohexadecanone;
cycloaliphatic aldehydes, such as 2,4-dimethyl-3-cyclohexenecarbaldehyde; 2-methyl-4-(2,2,6-trimethyl-cyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde; cycloaliphatic ketones, such as 1-(3,3-dimethylcyclohexyl)-4-penten-1-one; 1-(5,5-dimethyl-2-cyclohexen-1-yl)-4-penten-1-one; 2,3,8,8-tetramethyl-1,2,3,4,5,6,7,8-octahydro-2-naphthalenyl methyl ketone; methyl-2,6,10-trimethyl-2,5,9-cyclododecatrienylketone; tert-butyl-(2,4-dimethyl-3-cyclohexen-1-yl)ketone; esters of cyclic alcohols, such as 2-tert-butylcyclohexyl acetate; 4-tert-butylcyclohexyl acetate; 2-tert-pentylcyclohexyl acetate; 4-tert-pentylcyclohexyl acetate; decahydro-2-naphthyl acetate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5- or -6-indenyl acetate; 4,7-methano-3a,4,5,6,7,7a-hexahydro-5- or -6-indenyl propionate; 4,7-methano-3a,4,5,6,7,7a-hexahydro-5- or -6-indenyl isobutyrate; 4,7-methanooctahydro-5- or -6-indenyl acetate;
esters of cycloaliphatic carboxylic acids, such as allyl 3-cyclohexylpropionate; allyl cyclohexyloxyacetate; methyl dihydrojasmonate; methyl jasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexenecarboxylate; ethyl 2-methyl-1,3-dioxolane-2-acetate;
aromatic hydrocarbons, such as styrene and diphenylmethane;
araliphatic alcohols, such as benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenyl-ethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;
esters of araliphatic alcohols and aliphatic carboxylic acids, e.g. benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; α-trichloromethyl benzyl acetate; a,a-dimethylphenylethyl acetate; a,a-dimethylphenylethyl butyrate; cinnamyl acetate; 2-phenoxyethyl isobutyrate; 4-methoxy benzyl acetate;
araliphatic ethers, such as 2-phenylethyl methyl ether; 2-phenylethyl isoamyl ether; 2-phenylethyl 1-ethoxyethyl ether; phenylacetaldehyde dimethyl acetal; phenylacetaldehyde diethyl acetal; hydratropaldehyde dimethyl acetal; phenylacetaldehyde-glycerol acetal; 2,4,6-trimethyl-4-phenyl-1,3-dioxane; 4,4a,5,9b-tetrahydroindeno[1,2-d]-m-dioxin; 4,4a,5,9b-tetrahydro-2,4-dimethylindeno[1,2-d]-m-dioxin;
aromatic and araliphatic aldehydes, such as benzaldehyde; phenylacetaldehyde; 3-phenylpropanal; hydratropaaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-tert-butylphenyl)propanal; 3-(4-tert-butylphenyl)propanal; cinnamaldehyde; a-butylcinnamaldehyde; a-amylcinnamaldehyde; a-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal; aromatic and araliphatic ketones, such as acetophenone; 4-methylacetophenone; 4-methoxyacetophenone; 4-tert-butyl-2,6-dimethylacetophenone; 4-phenyl-2-butanone; 4-(4-hydroxyphenyl)-2-butanone; 1-(2-naphthalenyl)ethanone; benzophenone; 1,1,2,3,3,6-hexamethyl-5-indanylmethyl ketone; 6-tert-butyl-1,1-dimethyl-4-indanylmethyl-ketone; 1-[2,3-dihydro-1,1,2,6-tetramethyl-3-(1-methylethyl)-1H-5-indenyl]ethanone; 5′,6′,7′,8′-tetrahydro-3′,5,5′,6′,8′,8′-hexamethyl-2-acetonaphthone; aromatic and araliphatic carboxylic acids and their esters, such as benzoic acid; phenylacetic acid; methyl benzoate; ethyl benzoate; hexyl benzoate; benzyl benzoate; methyl phenyl acetate; ethyl phenyl acetate; geranyl phenyl acetate; phenylethyl phenyl acetate; methyl cinnamate; ethyl cinnamate; benzyl cinnamate; phenylethyl cinnamate; cinnamyl cinnamate; allylphenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;
nitrogen-containing aromatic compounds, such as 2,4,6-trinitro-1,3-dimethyl-5-tert-butylbenzene; 3,5-dinitro-2,6-dimethyl-4-tert-butyl acetophenone; cinnamic acid nitrile; 5-phenyl-3-methyl-2-pentenoic acid nitrile; 5-phenyl-3-methylpentanoic acid nitrile; methyl anthranilate; methyl N-methyl anthranilate; Schiff bases of methyl anthranilate with 7-hydroxy-3,7-dimethyloctanal, 2-methyl-3-(4-tert-butylphenyl)propanal or 2,4-dimethyl-3-cyclohexenecarbaldehyde; 6-isopropylquinoline; 6-isobutylquinoline; 6-sec-butylquinoline; indole; skatole; 2-methoxy-3-isopropylpyrazine; 2-isobutyl-3-methoxypyrazine; 4-(4,8-dimethyl-3,7-nonadienyl)-pyridine;
phenols, phenyl ethers and phenyl esters, such as estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresylphenyl acetate; from the group of heterocyclic compounds such as 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;
lactones, such as 1,4-octanolide; 3-methyl-1,4-octanolide; 1,4-nonanolide; 1,4-decanolide; 8-decene-1,4-olide; 1,4-undecanolide; 1,4-dodecanolide; 1,5-decanolide; 1,5-dodecanolide; 1,15-pentadecanolide; cis- and trans-11-pentadecene-1,15-olide; cis- and trans-12-pentadecene-1,15-olide; 1,16-hexadecanolide; 9-hexadecene-1,16-olide; 10-oxa-1,16-hexadecanolide; 11-oxa-1,16-hexadecanolide; 12-oxa-1,16-hexadecanolide; ethylene-1,12-dodecanedioate; ethylene-1,13-tridecanedioate; coumarin; 2,3-dihydrocoumarin; octahydrocoumarin;
   as well as the stereoisomers, enantiomers, positional isomers, diastereomers, cis/trans isomers or epimers of the substances mentioned above.

Of the aforementioned individual fragrances or individual odorants which can be encapsulated in the sense of the present invention, fragrances or odorants which have an aldehyde, carboxylic acid or ester functionality are particularly preferred for use.

Aldehydic fragrances or odorants, which also include the corresponding acetals as well as esters and lactones, can be divided into the following groups, namely
(i) aliphatic aldehydes and their acetals;
(ii) cycloaliphatic aldehydes;
(iii) aromatic or araliphatic aldehydes;
(iv) aliphatic, aromatic or araliphatic esters; and
(v) Lactones;
and their respective mixtures.

The above-mentioned fragrances or odorants with aldehyde, carboxylic acid or ester functionality and mixtures thereof are selected from one or more of the following groups:
aliphatic aldehydes and their acetals, such as hexanal; heptanal; octanal; nonanal;
decanal; undecanal; dodecanal; tridecanal; 2-methyloctanal; 2-methylnonanal; (f)-2-hexenal; (Z)-4-heptenal; 2,6-dimethyl-5-heptenal; 10-undecenal; (f)-4-decenal; 2-dodecenal; 2,6,10-trimethyl-5,9-undecadienal; heptanal-diethyl acetal; 1,1-dimethoxy-2,2,5-trimethyl-4-hexene; citronellyloxyacetaldehyde;
cycloaliphatic aldehydes, such as 2,4-dimethyl-3-cyclohexenecarbaldehyde; 2-methyl-4-(2,2,6-trimethyl-cyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde; aromatic and araliphatic aldehydes, such as benzaldehyde; phenylacetaldehyde; 3-phenylpropanal; hydratropaaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-te/t-butylphenyl)propanal; 3-(4-te/t-butylphenyl)propanal; cinnamaldehyde; α-butylcinnamaldehyde; α-amylcinnamaldehyde; α-hexylcinnamaldehyde; 3-methyl-5-phenylpentanal; 4-methoxybenzaldehyde; 4-hydroxy-3-methoxybenzaldehyde; 4-hydroxy-3ethoxybenzaldehyde; 3,4-methylenedioxybenzaldehyde; 3,4-dimethoxybenzaldehyde; 2-methyl- 3-(4-methoxyphenyl)-propanal; 2-methyl-3-(4-methylenedioxyphenyl)-propanal;
aliphatic carboxylic acid esters, such as (E)- and (Z)-3-hexenyl formate; ethyl acetoacetate; isoamyl acetate; hexyl acetate; 3,5,5-trimethylhexyl acetate; 3-methyl-2-butenyl acetate; (f)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3- hexenyl isobutyrate; hexyl crotonate; ethyl isovalerate; ethyl 2-methyl pentanoate; ethyl hexanoate; allyl hexanoate; ethyl heptanoate; allyl heptanoate; ethyl octanoate; ethyl (E,Z)-2,4-decadienoate; methyl 2-octinate; methyl 2-noninate; allyl 2-isoamyloxyacetate; methyl 3,7-dimethyl 2,6-octadienoate;
esters of cyclic alcohols, such as 2-te/t-butyl cyclohexyl acetate; 4-te/t-butyl cyclohexyl acetate; 2-ieri-pentyl cyclohexyl acetate; 4-te/t-pentyl cyclohexyl acetate; decahydro-2-naphthyl acetate; 3-pentyltetrahydro-2/-/-pyran-4-yl acetate; decahydro-2,5,5,8a-tetramethyl-2-naphthyl acetate; 4,7-methano-3a,4,5,6,7,7a-hexahydro-5- or -6-indenyl acetate; 4,7-methano-3a,4,5,6,7,7a-hexahydro-5- or -6-indenyl propionate; 4,7-methano-3a,4,5,6,7,7a-hexahydro-5- or -6-indenyl isobutyrate; 4,7-methanooctahydro-5- or -6-indenyl acetate;
esters of araliphatic alcohols and aliphatic carboxylic acids, such as benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; a-trichloromethyl benzyl acetate; α,α-dimethylphenylethyl acetate; α,α-dimethylphenylethyl butyrate; cinnamyl acetate; 2-phenoxyethyl isobutyrate; 4-methoxybenzyl acetate;
esters of cycloaliphatic carboxylic acids, such as allyl 3-cyclohexylpropionate; allyl cyclohexyloxyacetate; methyl dihydrojasmonate; methyl jasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexenecarboxylate; ethyl 2-methyl-1,3-dioxolane-2-acetate;
aromatic and araliphatic carboxylic acid esters, such as methyl benzoate; ethyl benzoate; hexyl benzoate; benzyl benzoate; methyl phenyl acetate; ethyl phenyl acetate; geranyl phenyl acetate; phenyl ethyl phenyl acetate; methyl cinnamate; ethyl cinnamate; benzyl cinnamate; phenyl ethyl cinnamate; cinnamyl cinnamate; allyl phenoxy acetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate.

Listed below are aldehydes, acetals, esters and lactones with their commercial names, which are particularly preferred as representatives of groups (i) to (v) for the purposes of the method according to the invention:
aldehydes: 2-Methylpentanal; Aldehyd C 12 MNA HM; Aldehyde C 4; Aldehyde C 5; Aldehyde C 6; Aldehyde C 7; Aldehyde C 8; Aldehyde C 9; Aldehyde C 10; Aldehyde C 11 ISO; Aldehyde C 11 MOA PURE; Aldehyde C 11 UNDECANAL; Aldehyde C 11 UNDEYLENIC; Aldehyde C 12; Aldehyde C 12 MNA; Aldehyde C 13; ALDEHYDE MADARINE; AMYL CINNAMIC ALDEHYDE ALPHA; ANISALALDEHYDE-O; ANISYL ALDEHYDE; BENZALDEHYDE NAT.BERGAMAL; BORONAL; BOURGENOAL; CAMPHONELIC ALDEHYDE; CITRAL; CITRONELLAL HM; CITRONELLYL OXYACET ALDEHYDE; CITRYLAL; CITROYLAL E HM; CORTEX ALDEHYDE; CORTEX ALDEHYDE 50 PCT PEMOSA; CROTONIC ALDEHYDE; CUMINAL ALDEHYDE; CYCLAMEN ALDEHYDE; DECADIENAL TRANS, TRANS-2,4, DECANAL CIS-4; DECANAL TRANS-2; DECANAL TRANS-2 NAT; DECANAL TRANS-4; DECANAL-9,1; DODECANIENAL 2,6; DODECANAL TRANS-2; DUPICAL; EPOXYDECENAL-4,5-2 10% TRI; ETHYL HEXANAL; FARENAL®; FLORHYDRAL; GERALDEHYDE; HELIONAL; HELIOPAN; HELIOTROPIN; HEPTADIENAL TRANS, TRANS,2-4; HEPTENAL CIS-4; HEPTENAL TRANS-2; HEXENAL TRANS-2; HEXYL CINNAMIC ALDEHYDE ALPHA; HYDRATROPIC ALDEHYDE; HYDROXY CITRONELLAL; INTRELEVEN ALDEHYDE SPEC.; ISONONYL ALDEHYDE; ISOVALERIC ALDEHYDE; LEMON ALDEHYDE H&R JS I; LILIAL; LINOLAL; LYRAL; MAJANTAL; MANDRINAL; MANDRAINE ALDEHYDE 10% IN TEC BHT; MEFRANAL; MELONAL®; METHODY CITRONELLAL; METHYL BUTYRALDEHYDE; METHYL CINNAMIC ALDEHYDE ALPHA; METHYL PHENYLPENTENAL-4,2,2; METHYL THIO PROPANAL-3; METHYL TRIDECANAL-12 10% VT; METHYL-3-BUTEN-2-AL; METHYL-5-PHENYL-2-HEXEN-2-AL; MUGENAL 50 DPG; NEOCYCLO CITRAL; NONADIENAL; TRANS, CIS-2,6; NONENAL CIS-6; NONENAL TRANS-2; ONCIDAL® 3/060251; PENTENAL TRANS-2; PERILLA ALDEHYDE; PHENYLACETALDEHYDE; PHENYLBUTENALTRANS-2,2; PHENYLPROPYLALDEHYDE; PINOACET ALDEHYDE; PROFRANESAL; PROPIONALALDEHYDE 2-(P-TOLYL); PROPIONIC ALDEHYDE; PS-IRALDEIN X NEU; SAFRANAL; SALICYLIC ALDEHYDE FG; SILVIAL; TETRAHYDRO CITRAL; TIGLIC ALDEHYDE-2,2; TOLYL ALDEHYDE PARA FG; TRIDECENAL TRANS-2; TRIFERNAL; UNDECADIENAL-2,4; UNDECENAL TRANS-2; VERNALALDEHYDE; VERTOCITRAL; VERTOMUGAL; VERTIPRENAL; VETRAL ROH; ZIMTALDEHYDE NAT. HM; acetals: FLOROPAL; HEPTANAL DIETHYL ACETAL; NONANDIENAL DIETHYL ACETAL; OKOUMAL; PHENYLACET ALD. GLYCERIN ACETAL; PHENYLACETALDEYHDEDIMETHYLACETAL; esters: JASMAL; JESSEMAL; KHARISMAL; TIRAMISONE®.

In a further variant of the method according to the invention, flavoring substances can also be encapsulated as a core material in the form of a single flavor, wherein the core material comprises at least one single flavoring substance or mixtures thereof as the active ingredient.

Typical examples of flavoring substances or flavors which can be encapsulated in the sense of the invention are selected from the group consisting of: acetophenone; allyl capronate; alpha-ionone; beta-ionone; anisaldehyde; anisyl acetate; anisyl formate; benzaldehyde; benzothiazole; benzyl acetate; benzyl alcohol; benzyl benzoate; beta-ionone; butyl butyrate; butyl capronate; butylidene phthalide; carvone; camphene; caryophyllene; cineole; cinnamyl acetate; citral; citronellol; citronellal; citronellyl acetate; cyclohexyl acetate; cymol; damascone; decalactone; dihydrocoumarin; dimethyl anthranilate; dimethyl anthranilate; dodecalactone; ethoxyethyl acetate; ethyl butyric acid; ethyl butyrate; ethyl caprinate; ethyl capronate; ethyl crotonate; ethyl furaneol; ethyl guaiacol; ethyl isobutyrate; ethyl isovalerate; ethyl lactate; ethyl methyl butyrate; ethyl propionate; eucalyptol; eugenol; ethyl heptylate; 4-(p-hydroxyphenyl)-2-butanone; gamma-decalactone; geraniol; geranyl acetate; geranyl acetate; grapefruit aldehyde; methyl dihydrojasmonate (e. g. Hedion®); heliotropin; 2-heptanone; 3-heptanone; 4-heptanone; trans-2-heptenal; cis-4-heptenal; trans-2-hexenal; cis-3-hexenol; trans-2-hexenoic acid; trans-3-hexenoic acid; cis-2-hexenyl acetate; cis-3-hexenyl acetate; cis-3-hexenyl capronate; trans-2-hexenyl capronate; cis-3-hexenyl formate; cis-2-hexyl acetate; cis-3-hexyl acetate; trans-2-hexyl acetate; cis-3-hexyl formate; para-hydroxybenzylacetone; isoamyl alcohol; isoamyl isovalerate; isobutyl butyrate; isobutyraldehyde; isoeugenol methyl ether; isopropyl methyl thiazole; lauric acid; leavulinic acid; linalool; linalool oxide; linalyl acetate; menthol; menthofuran; methyl anthranilate; methyl butanol; methyl butyric acid; 2-methyl butyl acetate; methyl capronate; methyl cinnamate; 5-methyl furfural; 3,2,2-methyl cyclopentenolone; 6,5,2-methyl heptenone; methyl dihydrojasmonate; methyl jasmonate; 2-methyl methyl butyrate; 2-methyl 2-pentenolic acid; methyl thiobutyrate; 3,1-methyl thiohexanol; 3-methyl thiohexyl acetate; nerol; neryl acetate; trans,trans-2,4-nonadienal; 2,4-nonadienol; 2,6-nonadienol; 2,4-nonadienol; nootkatone; delta-octalactone; gamma-octalactone; 2-octanol; 3-octanol; 1,3-octenol; 1-octyl acetate; 3-octyl acetate; palmitic acid; paraldehyde; phellandrene; pentanedione; phenylethyl acetate; phenylethyl alcohol; phenylethyl alcohol; phenylethyl isovalerate; piperonal; propionaldehyde; propyl butyrate; pulegone; pulegol; sinensal; sulfurol; terpinene; terpineol; terpinolene; 8,3-thiomenthanone; 4,4,2-thiomethylpentanone; thymol; delta-undecalactone; gamma-undecalactone; valencene; valeric acid; vanillin; acetoin; ethylvanillin; ethylvanillin isobutyrate (3-ethoxy-4-isobutyryloxybenzaldehyde); 2,5-dimethyl-4-hydroxy-3(2H)-furanone and its derivatives (preferably homofuraneol (2-ethyl-4-hydroxy-5-methyl-3(2H)-furanone), homofuronol (2-ethyl-5-methyl-4-hydroxy-3(2H)-furanone and 5-ethyl-2-methyl-4-hydroxy-3(2H)-furanone); maltol and maltol derivatives (preferably ethylmaltol); coumarin and coumarin derivatives; gamma-lactones (preferably gamma-undecalactone, gamma-nonalactone, gamma-decalactone); delta-lactones (preferably 4-methyldeltadecalactone, massoilactone, deltadecalactone, tuberolactone); methyl sorbate; divanillin; 4-hydroxy-2(or 5)-ethyl-5(or 2)-methyl-3(2H)furanone; 2-hydroxy-3-methyl-2-cyclopentenone; 3-hydroxy-4,5-dimethyl-2(5H)-furanone; acetic acid isoamyl ester; butyric acid ethyl ester; butyric acid n-butyl ester; butyric acid isoamyl ester; 3-methyl-butyric acid ethyl ester; n-hexanoic acid ethyl ester; n-hexanoic acid allyl ester; n-hexanoic acid n-butyl ester; n-octanoic acid ethyl ester; ethyl 3-methyl-3-phenyl glycidate; ethyl 2-trans-4-cis-decadienoate; 4-(p-hydroxyphenyl)-2-butanone; 1,1-dimethoxy-2,2,5-trimethyl-4-hexane; 2,6-dimethyl-5-hepten-1-al; phenylacetaldehyde; 2-methyl-3-(methylthio)furan; 2-methyl-3-furanethiol; bis(2-methyl-3-furyl)disulfide; furfuryl mercaptan; methional; 2-acetyl-2-thiazoline; 3-mercapto-2-pentanone; 2,5-dimethyl-3-furanethiol; 2,4,5-trimethylthiazole; 2-acetylthiazole; 2,4-dimethyl-5-ethylthiazole; 2-acetyl-1-pyrroline; 2-methyl-3-ethylpyrazine; 2-ethyl-3,5-dimethylpyrazine; 2-ethyl-3,6-dimethylpyrazine; 2,3-diethyl-5-methylpyrazine; 3-isopropyl-2-methoxypyrazine; 3-isobutyl-2-methoxypyrazine; 2-acetylpyrazine; 2-pentylpyridine; (E,E)-2,4-decadienal; (E,E)-2,4-nonadienal; (E)-2-octenal; (E)-2-nonenal; 2-undecenal; 12-methyltridecanal; 1-penten-3-one; 4-hydroxy-2,5-dimethyl-3(2H)-furanone; guaiacol; 3-hydroxy-4,5-dimethyl-2(5H)-furanone; 3-hydroxy-4-methyl-5-ethyl-2(5H)-furanone; cinnamaldehyde; cinnamyl alcohol; methyl salicylate; isopulegol and the stereoisomers, enantiomers, positional isomers, diastereomers, cis/trans isomers or epimers of these substances not explicitly mentioned herein; and mixtures of the aforementioned substances.

In an alternative embodiment according to the present invention, in the plant protein-based microcapsules according to the invention, a fragrance or odorant mixture or a perfume oil or an aroma mixture or an aroma is used as the active ingredient to be encapsulated or as the core material. These are compositions that contain at least one fragrance or odorant or a flavoring substance. Such compositions, in particular fragrance or odorant mixtures or perfume oils, preferably comprise two, three, four, five, six, seven, eight, nine, ten or more fragrance or odorant substances. The fragrance or odorant mixtures or perfume oils are preferably selected from the group of extracts from natural raw materials, such as etheric oils, concretes, absolutes, resins, resinoids, balsams, tinctures such as e.g. amber tincture; amyris oil; angelica seed oil; angelica root oil; anise oil; valerian oil; basil oil; tree moss absolute; bay oil; mugwort oil; benzöresin; bergamot oil; beeswax absolute; birch tar oil; bitter almond oil; savory oil; bucco leaf oil; cabreuva oil; cade oil; calmus oil; camphor oil; cananga oil; cardamom oil; cascarilla oil; cassia oil; cassie absolute; castoreum absolute; cedar leaf oil; cedarwood oil; cistus oil; citronella oil; citron oil; copaiva balsam; copaiva balsam oil; coriander oil; costus root oil; cumin oil; cypress oil; davana oil; dill oil; dill seed oil; eau de brouts absolute; oak moss absolute; elemi oil; tarragon oil; eucalyptus citriodora oil; eucalyptus oil; fennel oil; spruce needle oil; galbanum oil; galbanum resin; geranium oil; grapefruit oil; guaiac wood oil; gurjun balsam; gurjun balsam oil, helichrysum absolute; helichrysum oil; ginger oil; iris root absolute; iris root oil; jasmine absolute; calamus oil; blue chamomile oil; roman chamomile oil; carrot seed oil; cascarilla oil; pine needle oil; spearmint oil; caraway oil; labdanum oil; labdanum absolute; labdanum resin; lavandin absolute; lavandin oil; lavender absolute; lavender oil; lemongrass oil; lovage oil; lime oil distilled; lime oil pressed; linal oil; litsea cubeba oil; bay leaf oil; mace oil; marjoram oil; mandarin oil; masso bark oil; mimosa absolute; musk grain oil; musk tincture; muscatel sage oil; nutmeg oil; myrrh absolute; myrrh oil; myrtle oil; clove leaf oil; clove blossom oil; neroli oil; olibanum absolute; olibanum oil; opopanax oil; orange blossom absolute; orange oil; origanum oil; palmarosa oil; patchouli oil; perilla oil; peru balsam oil; parsley leaf oil; parsley seed oil; petitgrain oil; peppermint oil; pepper oil; allspice oil; pine oil; poley oil; rose absolute; rosewood oil; rose oil; rosemary oil; sage oil dalmatian; sage oil spanish; sandalwood oil; celery seed oil; spiked lavender oil; star anise oil; styrax oil; tagetes oil; fir needle oil; tea tree oil; turpentine oil; thyme oil; tolu balsam; tonka absolute; tuberose absolute; vanilla extract; violet leaf absolute; verbena oil; vetiver oil; juniper berry oil; wine yeast oil; wormwood oil; wintergreen oil; ylang oil; hyssop oil; civet absolute; cinnamon leaf oil; cinnamon bark oil; and fractions thereof or ingredients isolated therefrom. ingredients isolated therefrom.

Most preferably, in the process according to the invention, fragrances or odorants or flavorings are used which are selected from the group consisting of: AGRUMEX LC; AGRUNITRIL; ALDEHYD C11 UNDECYLENIC; ALDEHYD C12 LAURIN; ALDEHYD C12 MNA; ALDEHYD C14 SOG; ALDEHYD C16 SOG.; ALLYLAMYLGLYCOLAT; ALLYLCAPRONAT; ALLYLCYCLOHEXYLPROPIONAT; ALLYLHEPTYLAT; AMBROCENIDE® 10 TEC; AMBROCENIDE® Krist. 10% IPM; AMBROXIDE; ANETHOL NAT. EX STERNANIS; ANISALDEHYD REIN; APRIFLOREN®; BENZYLACETON; BENZYLSALICYLAT; BORNEOL L/ISOBORNEOL 65/35; BUCCOBLAETTEROEL; CITRONELLOL 950; CLONAL; CYCLOHEXYLSALICYLAT; CYMOL PARA SUPRA; DAMASCONE DELTA; DIHYDROMYRCENOL; DIMETHYLBENZYLCARBINYLBUTYRAT; DYNASCONE; ETHYLENEBRASSYLAT; ETHYLMETHYLBUTYRAT-2; ETHYLSAFRANAT; EUCALYPTOL NAT.; EUKALYPTUSOEL GLOBULUS 80/85%; EUGENOL NAT.; FARENAL®; FENCHELOEL AROMA TYP SUESS NAT.; FILBERTONE 10% IPM; FILBERTONE; FLOROPAL; GALBASCONE; GERANIOL 60; GLOBANONE®; HEDION; HERBAFLORAT; HERBANATE; HERBYLPROPIONAT; HEXENYLACETAT CIS-3; HEXENYLSALICYLAT CIS-3; HEXYLACETAT; HEXYLACETAT S; HEXYLISOBUTYRAT; HEXYLSALICYLAT; ISOAMYLBUTYRAT; ISOBORNYLACETAT; ISOPROPYLMETHYLBUTYRAT-2; ISORALDEIN 70; JAVANOL; KAMPFER DL; KRESOLMETHYLETHER P(CR<10 PPM); LEMONILE; LIGUSTRAL; LILIAL; LINALOOL; MANZANATE; MELONAL; METHYLHEPTINCARBONAT; METHYLOCTINCARBONAT; MUSCENONE; NEOCYCLOCITRAL; NEROLIN BROMELIA; NEROLIN YARA YARA KRIST.; NEROLIONE; NORLIMBANOL; ORANGENOEL; ORIVONE; OZONIL; PATCHOULIOEL ENTF.; PFLANZENOL TRIGLYCERID; PHELLANDREN FRAKTION EX EUKALYPTUSOEL; PHENIRAT®; PHENYLETHYLACETAT; ROSENOXID HIGH CIS; SANDRANOL®; STYROLYLACETAT; SULTANENE®; TERPINEN GAMMA; TETRAHYDROLINALOOL; TIMBERSILK; TRIETHYLCITRAT; UNDECAVERTOL; VERTOCITRAL; VERTOFIX; YSAMBER® K and mixtures of the above active ingredients.

Exemplary cooling agents used as hydrophobic active ingredients in the preparation of the microcapsules according to the invention include one or more of menthol and menthol derivatives (for example, L-menthol, D-menthol, racemic menthol, isomenthol, neoisomenthol, neomenthol), menthyl ether (for example (1-menthoxy)-2-propanediol, (1-menthoxy)-2-methyl-1,2-propanediol, 1-menthyl methyl ether), menthyl esters (for example menthyl formate, menthyl acetate, menthyl isobutyrate, menthyl lactate, L-menthyl L-lactate, L-menthyl D-lactate, menthyl (2-methoxy) acetate, menthyl (2-methoxyethoxy) acetate, menthyl pyroglutamate), menthyl carbonates (for example menthyl propylene glycol carbonate, menthyl ethylene glycol carbonate, menthyl glycerol carbonate or mixtures thereof), the semiester of menthol with a dicarboxylic acid or derivatives thereof (e.g. monomenthyl succinate, monomenthyl glutarate, monomenthyl malonate, O-menthylsuccinate-N,N-(dimethyl) amide, O-menthylsuccinamide), menthanecarboxamides (for example menthanecarboxylic acid-N-ethylamide [WS3], N-alpha-(methanecarbonyl) glycine ethyl ester [WS5], menthanecarboxylic acid-N-(4-cyanophenyl)-amide, menthanecarboxylic acid N-(alkoxyalkyl)amide), menthone and menthone derivatives (for example L-menthonglycerol ketal), 2,3-dimethyl-2-(2-propyl)-butanoic acid derivatives (for example 2,3-dimethyl-2-(2-propyl)-butanoic acid N-methylamide [WS23]), isopulegol or its esters (1-(−)-isopulegol, 1-(−)-isopulegol acetate), menthan derivatives (for example p-menthane-3,8-diol), cubebol or synthetic or natural mixtures containing cubebol, pyrrolidone derivatives of cycloalkyldione derivatives (for example 3-methyl)-2-(1-pyrrolidinyl)-2-cyclopenten-1-one) or tetrahydropyrimidin-2-ones (for example icilin or related compounds as described in WO 2004/026840). Further cooling agents are menthol (L-menthol, D-menthol, racemic menthol, isomenthol, neoisomenthol, neomenthol), L-menthyl methyl ether, menthyl formate, menthyl acetate), menthone, isopulegol, L-(−)-isopulegol acetate) and cubebol, which have a cooling flavor effect. Suitable cooling agents are well known in the art and are described, for example, in US 2017/216802 (A1), US 2010/273887 (A1), EP 2 033 688 (A2) and EP 1 958 627 (A2).

In an alternative variant, a TRPV1 or a TRPV3 modulator is used in the plant protein-based microcapsules according to the invention as the active ingredient to be encapsulated or as the core material. TRPV1 and TRPV3 modulators are known in the prior art and refer to TRP channels (Transient Receptor Potential channels) of the vanilloid (TRPV) subfamily. TRPV1 modulators impart a pungent taste and the hot sensation associated with capsaicin and piperine. The TRPV3 protein belongs to the family of non-selective cation channels that function in a variety of processes, including temperature sensation and vasoregulation. The TRPV3 channel is directly activated by various natural compounds such as carvacrol, thymol and eugenol. Some other monoterpenoids, which either cause a sensation of warmth or are skin sensitizers, can also open the channel. Monoterpenoids also induce agonist-specific desensitization of TRPV3 channels in a calcium-independent manner.

In a further variant, in the plant protein-based microcapsules according to the invention, an active substance is used as the active substance to be encapsulated or as the core material, which is selected from the group consisting of substances which cause a sharp taste or a sensation of warmth or heat on the skin or mucous membranes or a tingling or fizzing sensation in the mouth or throat, or active substances with a pungent or acrid or astringent effect.

The heat-inducing or pungent active ingredients are preferably selected from the group consisting of: paprika powder, chili pepper powder, extracts from paprika, extracts from pepper, extracts from chili pepper, extracts from ginger roots, extracts from grains of paradise (Aframomum melegueta), extracts from paracress (Jambu oleoresin; Spilanthes acmella, resp. Spilanthes oleracea), extracts from Japanese pepper (Zanthoxylum piperitum), extracts from Kaempferia galanga, extracts from Alpinia galanga, extracts from water pepper (Polygonium hydropiper), capsaicinoids, in particular capsaicin, dihydrocapsaicin or nonivamide; gingerols, in particular gingerol-[6], gingerol-[8], or gingerol-[10]; shogaols, in particular shogaol-[6], shogaol-[8], shogaol-[10]; gingerdiones, in particular gingerdione-[6], gingerdione-[8] or gingerdione-[10]; paradoles, in particular paradol-[6], paradol-[8] or paradol-[10]; dehydrogingerdiones, in particular dehydrogingerdione-[6], dehydrogingerdione-[8] or dehydrogingerdione-[10]; piperine; piperine derivatives; ethyl 2-(4-hydroxy-3-methoxy-phenyl)acetate and 3-phenylpropyl-2-(4-hydroxy-3-methoxy-phenyl)acetate and mixtures thereof.

The active ingredients which can be perceived as pungent or acrid are preferably selected from the group consisting of: aromatic isothiocyanates, in particular phenylethyl isothiocyanate, allyl isothiocyanate, cyclopropyl isothiocyanate, butyl isothiocyanate, 3-methylthiopropyl isothiocyanate, 4-hydroxybenzyl isothiocyanate, 4-methoxybenzyl isothiocyanate and mixtures thereof.

The tingling agents are preferably selected from the group consisting of 2E,4E-decadienoic acid-N-isobutylamide (trans-pellitorin), in particular those as described in WO 2004/043906; 2E,4Z-decadienoic acid-N-isobutylamide (cis-pellitorin), in particular those as described in WO 2004/000787; 2Z,4Z-decadienoic acid-N-isobutylamide; 2Z,4E- decadienoic acid-N-isobutylamide; 2E,4E-decadienoic acid-N-([2S]-2-methylbutyl)amide; 2E,4E-decadienoic acid-N-([2S]-2-methylbutyl)amide; 2E,4E-decadienoic acid-N-([2R]-2-methylbutylamide); 2E,4Z-decadienoic acid-N-(2-methylbutyl)amide; 2E,4E-decadienoic acid-N-piperide (achilleamide); 2E,4E-decadienoic acid-N-piperide (sarmentin); 2E-decenoic acid-N-isobutylamide; 3E-decenoic acid-N-isobutylamide; 3E-nonenoic acid-N-isobutylamide; 2E,6Z,8E-decatrienoic acid-N-isobutylamide (spilanthol); 2E,6Z,8E-decatrienoic acid-N-([2S]-2-methylbutyl)amide (homospilanthol); 2E,6Z,8E-decatrienoic acid-N-([2R]-2-methylbutyl)amide; 2E-decene-4-yonic acid-N-isobutylamide; 2Z-decene-4-yonic acid-N-isobutylamide; 2E,6Z,8E,10E-dodecatetraenoic acid-N-(2-methylpropyl)amide (alpha-sanshool); 2E,6Z,8E,10E-dodecatetraenoic acid-N-(2-hydroxy-2-methylpropyl)-amide (alpha-hydroxysanshool); 2E,6E,8E,10E-dodecatetraenoic acid-N-(2-hydroxy-2-methylpropyl)amide (gamma-hydroxysanshool); 2E,4E,8Z,10E, 12E-tetradeca-pentaenoic acid-N-(2-hydroxy-2-methylpropyl)amide (gamma-hydroxysanshool); 2E,4E,8E,10E,12E-tetradecapentaenoic acid-N-(2-hydroxy-2-methylpropyl)-amide-(gamma-hydroxyisosanshool); 2E,4E,8Z, 10E,12E-tetradecapentaenoic acid-N-(2-methyl-2-propenyl)amide (gamma-dehydrosanshool); 2E,4E,8Z,10E,12E-tetradecapentaenoic acid-N-(2-methylpropyl)-amide (gamma-sanshool); 2E,4E,8Z,11Z-tetradecatetraenoic acid-N-(2-hydroxy-2-methylpropyl)amide (bungeanool); 2E,4E,8Z,11E-tetradecatetraenoic acid-N-(2-hydroxy-2-methylpropyl)amide (isobungeanool); 2E,4E,8Z-tetradecatrienoic acid-N-(2-hydroxy-2-methylpropyl)amide (dihydrobungeanool) and 2E,4E-tetradecadienoic acid-N-(2-hydroxy-2-methylpropyl)-amide (tetrahydrobungeanool) and mixtures thereof.

Active ingredients with an astringent effect are preferably selected from the group consisting of: catechins, in particular epicatechins, gallocatechins, epigallocatechins as well as their respective gallic acid esters, in particular epigallocatechin gallate or epicatechin gallate, their oligomers (procyanidins, proanthocyanidins, prodelphinidins, procyanirins, thearubigenins, theogallins) as well as their C- and O-glycosides; dihydroflavonoids such as dihydromyricetin, taxifolin, and their C- and O-glycosides, flavonols such as myricetin, quercetin and their C- and O-glycosides such as quercetrin, rutin, gallic acid esters of carbohydrates such as tannin, pentagalloyl glucose or their reaction products such as elligatannin, aluminum salts, e.g. alum, and their mixtures.

In a further variant according to the first and/or second aspect of the present invention, biogenic principles can also be encapsulated as core material, wherein the core material comprises at least one biogenic principle or mixtures thereof.

Biogenic principles are active ingredients with biological activity, for example tocopherol, tocopherol acetate, tocopherol palmitate, ascorbic acid, carnotine, carnosine, caffeine, (deoxy)ribonucleic acid and its fragmentation products, p-glucans, retinol, bisabolol, allantoin, phytantriol, panthenol, AHA acids, amino acids, ceramides, pseudoceramides, essential oils, plant extracts and vitamin complexes.

In a further variant of the process according to the invention, substances for printing coatings for paper are also used as the active ingredient to be encapsulated or as the core material, as described in U.S. Pat. No. 2,800,457A, the disclosure of which in this regard is adopted in its entirety by reference to the present description.

The internal non-aqueous phase may contain, for example, 20 to 80 wt.- %, preferably 25 to 75 wt.- % and even more preferably 33 to 50 wt. % of the hydrophobic active ingredient to be encapsulated, 0.1 to 5 wt.- %, preferably 0.15 to 3.5 wt.- % and even more preferably 0.5 to 2.5 wt.- % crosslinking agent and, in addition to 100 wt.- %, a hydrophobic solvent, based on the total weight of the internal non-aqueous phase.

It is thus possible to achieve a high loading of the plant protein-based microcapsules according to the invention with active ingredient using the process according to the invention.

In a further step (ii) of the process according to the invention, an external aqueous phase is provided, comprising at least one plant protein,
and optionally at least one first polysaccharide and/or at least one further crosslinking agent and/or at least one polyhydroxyphenol and/or at least one protective colloid, and further optionally adjusting the pH of the aqueous phase to a pH below the isoelectric point of the plant protein.

The polyhydroxyphenol optionally added in step (ii) may also be a further crosslinking agent. The polyhydroxyphenol can also be a crosslinking agent itself. Optionally, the polyhydroxyphenol may alternatively or additionally be added in step (i). The polyhydroxyphenol is preferably also a tannin.

In a preferred embodiment of the invention, an external aqueous phase comprising at least one plant protein and at least one first polysaccharide can be provided in step (ii).

Particularly preferred is the plant protein in a case where in step (ii) an external aqueous phase is provided comprising at least one plant protein and at least one first polysaccharide, wherein the plant protein is preferably selected from the group consisting of pea protein, sunflower protein, hemp protein, pumpkin protein or soy protein.

Particularly preferably, in a case where in step (ii) an external aqueous phase comprising at least one plant protein and at least one first polysaccharide is provided, the at least one first polysaccharide is carboxymethylcellulose (CMC) and/or a pectin.

The pectin is preferably a highly esterified pectin, which preferably has a degree of esterification of at least 50%, particularly preferably >68%.

Suitable solvents for preparing the external aqueous phase are water or mixtures of water with at least one water-miscible organic solvent. Suitable organic solvents are, for example, glycerol, 1,2-propanediol, 1,3-propanediol, ethanediol, diethylene glycol, triethylene glycol and other analogs. Preferably, however, the solvent is water.

According to the present invention, the at least one plant protein or the at least one further plant protein is selected from the group consisting of protein isolates, plant proteins in the form of fractions, partial or complete hydrolysates or intermediates produced by physico-chemical processes or fermentative or enzymatic treatment of the proteins, in particular plant proteins of the group consisting of: cereals, in particular wheat, barley, rye, spelt, gluten, in particular wheat gluten, rapeseed, rice, potatoes, corn, soybeans, beans, chickpeas, lentils, lupins, peanuts, alfalfa, fava beans, peas, hemp, pumpkin and sunflowers, other proteins from edible plants, chitosan and mixtures thereof.

Particularly preferred is the at least one plant protein selected from the group consisting of hemp protein, pumpkin protein, soy protein, sunflower protein and pea protein. The at least one plant protein is particularly preferably hemp protein or pumpkin protein. These have particularly good stability. Alternatively, soy protein and pumpkin protein are also preferred due to their sensory properties.

Among the above-mentioned proteins, peas, hemp, pumpkin, soy and sunflower are particularly preferred. In combination with aliphatic polyisocyanates as crosslinking agents, these are particularly suitable for obtaining vegan microcapsules which are characterized by particularly high stability and good sensory properties.

The amino acids may be proteinogenic L-amino acids. These may be selected from the group consisting of L-alanine, L-arginine, L-asparagine, L-aspartic acid, L-cysteine, L-glutamine, L-glutamic acid, L-glycine, L-histidine, L-isoleucine, L-leucine, L-lysine, L-methionine, L-phenylalanine, L-proline, L-serine, L-threonine, L-tryptophan, L-tyrosine and L-valine.

The above-mentioned proteins also have the advantageous effect that they have an emulsifying effect. Due to their emulsifying effect, they contribute to the stabilization of the emulsion. Due to their more or less flexible structure and their differently charged areas within the molecule, proteins are amphiphilic and therefore surface-active. By modifying the proteins, for example by physical or chemical modification, the secondary and/or tertiary structure of the molecule can be changed. This can influence the emulsifying properties by changing the spatial availability of charged areas of the molecule or exposing amino acid side chains. Due to these advantageous properties, an additional emulsifier or a protective colloid can be dispensed with in the process according to the invention.

The proportion of the at least one protein, preferably pea protein, pumpkin protein, hemp protein, soy protein or sunflower protein in the external aqueous phase is in a range from 0.01 to 7.0 wt.- %, preferably in a range from 0.05 to 5.0 wt.- %, particularly preferably in a range from 0.07 to 2.5 wt. %, based on the total weight of the external aqueous phase.

According to a further aspect of the present invention, the at least one first polysaccharide and/or the at least one further polysaccharide may be selected from the group consisting of
indigestible fibers and dietary fibers, in particular insoluble dietary fibers, in particular cellulose, cellulose derivatives such as hydroxyethyl cellulose, in particular quaternized hydroxyethyl cellulose, carboxymethylcellulose (CMC) and microcrystalline cellulose (MCC), hemicelluloses, lichenin, chitin, chitosan, lignin, xanthan gum, vegetable fibers, in particular cereal fibers, potato fibers, apple fibers, citrus fibers, bamboo fibers, extracted sugar beet fibers; oat fibers and soluble dietary fibers, in particular inulin, especially native inulin, highly soluble inulin, granulated inulin, high performance inulin, pectins, alginates, agar, carrageenan, tragacanth, gum arabic, konjac gum, gurdlan (paramylon), guar gum, locust bean gum, xanthan gum, raffinose, xylose, polydextrose and lactulose;
starches, in particular starch from wheat, potatoes, maize, rice, tapioca and oats, chemically, mechanically and/or enzymatically modified starch; and starch derivatives, e.g. dextrins or maltodextrins, in particular dextrins and maltodextrins from wheat, potatoes, maize, rice and oats, in particular maltodextrins DE8-10, DE17-20, DE18-20, cyclodextrins, oligosaccharides, in particular oligofructose; and
sugar alcohols, in particular sorbitol, mannitol, isomalt, maltitol, maltilol syrup, lactitol, xylitol, erythritol;
gellan; glucose,
glycosaminoglycans, in particular hyaluronic acid
   and mixtures of the aforementioned polysaccharides.

Among the aforementioned polysaccharides, gum arabic and maltodextrins are particularly preferred. Most preferred are the maltodextrins DE8- 10 potato; DE17- 20 maize; DE8-10 maize; DE17- 20 potato and DE18- 20 wheat.

In a particularly advantageous further development of the invention, it may be provided that the at least one first polysaccharide and/or the at least one further polysaccharide is selected from the group consisting of hyaluronic acid, carragenaan, gellan gum, agar agar, alginate, xanthan gum. In this way, plant protein-based microcapsules with very good sensory properties are obtained.

In particular, it may be provided that the first polysaccharide and/or the at least one further polysaccharide comprises at least hyaluronic acid and/or carrageenan and/or gellan gum and/or agar agar and/or alginate and/or xanthan gum.

The proportion of the at least one polysaccharide in the external aqueous phase is in a range from 0.1 to 3.0 wt.- %, preferably in a range from 0.2 to 1.0 wt.- %, based on the total weight of the external aqueous phase. Most preferably, the at least one polysaccharide is used in a range from 0.25 to 0.75 wt.- %, based on the total weight of the external aqueous phase.

Preferably, the external aqueous phase is provided with both main components of the capsule shell, i.e. at least one protein and at least one polysaccharide. The combined use of protein and polysaccharide results in the formation of soluble or insoluble protein-polysaccharide complexes. The emulsion formed in this way is less prone to aggregate formation, so that the addition of a protective colloid or an additional emulsifier is not necessary in the process according to the invention.

The following combinations of protein and polysaccharide are particularly preferred for building up the capsule wall or capsule shell: pea protein and maltodextrin; hemp protein and maltodextrin; pumpkin protein and maltodextrin, sunflower protein and maltodextrin; soy protein and maltodextrin.

The previously described and exemplary components for the structure of the capsule wall, proteins and polysaccharides, are readily available from biological sources. They are also readily biodegradable as such.

In an alternative variant of the process according to the invention, the external aqueous phase is provided with only one of the main components of the capsule shell protein or polysaccharide. In this variant, the addition of the other main constituent polysaccharide or protein optionally takes place in process step (iv) and/or in step (vi) after emulsification/dispersion and before or with an addition of the catalyst, which optionally takes place in process step (v).

By using at least one protein and at least one polysaccharide, the content of crosslinking agent polyisocyanate in the capsule shell can be reduced compared to the microcapsules of the prior art, which have a high polyisocyanate content.

Surprisingly, however, this lower degree of cross-linking leads to stable microcapsules on the one hand and to microcapsules with better biodegradability on the other hand, as illustrated in the following embodiment examples.

A protective colloid can optionally be added to the external aqueous phase.

A protective colloid is a polymer system that prevents clumping (agglomeration, coagulation, flocculation) of the emulsified, suspended or dispersed components in suspension or dispersion. During solvation, protective colloids bind large quantities of water and generate high viscosities in aqueous solutions, depending on the concentration. During the production of oil-in-water emulsions, the hydrophobic part of the protective colloid attaches itself to the primary particles and turns its polar, i.e. hydrophilic, part of the molecule towards the aqueous phase. By attaching to the interface in this way, it lowers the interfacial tension and prevents agglomeration of the primary particles. It also stabilizes the emulsion and promotes the formation of comparatively smaller droplets and thus corresponding microcapsules.

In the process according to the invention, the protective colloid also has emulsifying properties in addition to the above-mentioned properties. If the protective colloid, such as carboxymethyl cellulose (CMC), acid-modified starch, polyvinyl alcohol, ammonium derivatives of polyvinyl alcohol, polystyrene sulfonates, polyvinyl pyrollidones, polyvinyl acrylates, has sufficient emulsifying properties, the use of an emulsifier in the downstream emulsifying/dispersing step (iii) can advantageously even be dispensed with in the process according to the invention.

The protective colloid used in the process according to the invention is selected from the group consisting of
diols, in particular ethanediol, 1,2-propanediol, 1,3-propanediol, 1,2-butanediol, isomeric butanediol, 1,2-pentanediol, 1,2-hexanediol, 1,2-octanediol, 1,2-decanediol, 1,2-dodecanediol, and
polyols, preferably triols, in particular glycerol and its ethoxylation and propoxylation products, trimethylolpropane and its ethoxylation and propoxylation products, polyvinyl alcohol (PVOH) and its derivatives, in particular ammonium- or sulphonate-functionalized polyvinyl alcohols, polyphenols, preferably 1,3,5-trihydroxybenzene, polysaccharides, in particular glucose, starches or chemically, mechanically and/or enzymatically modified starches, cellulose derivatives such as hydroxyethyl cellulose, in particular quaternized hydroxyethyl cellulose, and carboxymethyl cellulose,
polyvinylpyrrolidone, maleic acid vinyl copolymers, sodium lignosulfonates, maleic acid anhydride/styrene copolymers, ethylene/maleic acid anhydride copolymers, copolymers of ethylene oxide, propylene oxide and acid esters of polyethoxylated sorbitol, sodium dodecyl sulfate,
vegetable polymers, in particular gum arabic (Senegal and Seyal type), olibanum resin, shellac, lignin, chitosan, saponin
   and mixtures of the aforementioned compounds.

Starches, in particular modified starches or vegetable polymers, are naturally occurring substances which are biodegradable. In combination with the polyisocyanates described herein, biobased and biodegradable capsule shells can thus be provided by the present process. In the process according to the invention, the starch and the plant polymers therefore also act as so-called biocrosslinkers.

The starch used in the process according to the invention is selected from the group consisting of maize starch, potato starch, rye starch, wheat starch, barley starch, oat starch, rice starch, pea starch, tapioca starch and mixtures thereof.

The chemically modified starches are preferably acid-modified starches, alkali-modified starches, oxidized starches, acetylated starches, succinized starches or ocentyl-succinized starches.

Preferably, the external aqueous phase comprises at least one protective colloid selected from polyvinylpyrrolidones, polyvinyl alcohols, polyols, polyphenols or starches and mixtures thereof.

Even more preferably, polyols, polyphenols or starch, in particular modified starch, are used as protective colloid. Polyvinyl alcohol or its ammonium derivatives, 1,3,5- trihydroxybenzene, modified starch or carboxymethylcellulose are particularly preferably used as protective colloid for the production of the microcapsules according to the invention.

According to the present invention, combinations of two or more different protective colloids can also be used to produce the microcapsule according to the invention.

It has proved to be particularly advantageous in the process according to the invention if a combination of one of the above-mentioned protective colloids with starch as a further protective colloid is used in the external aqueous phase. Such a combination stabilizes the emulsion due to the high number of functional hydroxyl groups and, on the other hand, promotes a reaction between the protective colloid and the polyisocyanate(s), whereby the reaction equilibrium in the reaction of the protective colloids with the polyisocyanate(s) is shifted to the side of the products, i.e. the polyurethanes. The large number of functional hydroxyl groups in starch also enables the formation of particularly pronounced spatial cross-links.

Depending on the number of functional groups and/or the size of the protective colloid, the above-mentioned protective colloids have different reaction rates with the isocyanate groups of the at least one polyisocyanate. For example, glycerol reacts faster with the isocyanate groups than starch, for example, due to its size. Therefore, the crosslinking of the protective colloid with the isocyanate groups of the polyisocyanate can be controlled by the selection of the protective colloid.

Glycerol with starch or with modified starch or the combination of glycerol with quaternized hydroxyethyl cellulose or gum arabic type Seyal has proven to be a particularly advantageous combination; such a combination makes use of the previously described properties of both protective colloids: high reaction speed of glycerol on the one hand and number of polymerizable functional groups of the other protective colloid on the other.

The protective colloids used in the process according to the invention have a dual function in that, on the one hand, they act as a protective colloid and thus prevent the agglomeration of the emulsified, suspended or dispersed components, stabilize the subsequently formed emulsion, promote the formation of small droplets and stabilize the microcapsule dispersion ultimately formed.

The external aqueous phase is preferably prepared with stirring by successively adding the polysaccharide and/or the protein and optionally the protective colloid or vice versa to the external aqueous phase, or by adding the components simultaneously to the external aqueous phase.

To improve the solubility of the protein, the pH value of the external aqueous phase is optionally adjusted to a pH value below the isoelectric point of the protein, i.e. to a pH value that is lower than the isoelectric point of the protein used.

The isoelectric point is the pH value at which the isoelectric state is reached, i.e. at which the positive and negative charges of ampholytes or zwitterions (e.g. amino acids and proteins) are balanced. This value is a characteristic constant for each amino acid and depends on the pKₐ value of the functional group. In addition to amino acids, peptides and proteins also have an isoelectric point. Amino acids, and therefore also proteins, have the lowest water solubility at the isoelectric point.

Preferably, the pH value of the aqueous phase is adjusted to a pH value in the range from 2.0 to 7.0, even more preferably to a pH value in the range from 2.0 to 6.0 and most preferably to a slightly acidic pH value in the range from 3.0 to 5.0, depending on the isoelectric point of the protein used. Adjusting the pH to a pH value below the isoelectric point, i.e. to a pH value lower than the isoelectric point, of the protein has the advantage that the emulsifying properties and the solubility of the protein are greatest at such a pH value.

The pH value of the external aqueous phase is adjusted by adding an organic acid. For this purpose, an organic acid, for example formic acid or acetic acid, is added to the external aqueous phase before the emulsification step and a pH value in the above-mentioned ranges is set.

The internal non-aqueous phase, which comprises at least one crosslinking agent and at least one hydrophobic active ingredient, is emulsified or dispersed in the external aqueous phase in a further process step (iii) to form an oil-in-water emulsion/dispersion.

The oil-in-water emulsion is prepared by mixing the internal non-aqueous phase and the external aqueous phase. The weight ratio of internal non-aqueous phase to external aqueous phase is preferably in a range of 70:30 to 60:40, preferably in a range of 30:70 to 60:40.

In order to facilitate the formation of an emulsion or dispersion from an internal non-aqueous phase and an external aqueous phase, to stabilize the emulsion or dispersion formed and to prevent segregation of the internal non-aqueous (oily/organic/hydrophobic) phase and the external aqueous (hydrophilic) phase, a stabilizer and/or an emulsifier or an emulsifying aid is optionally added to the emulsion or dispersion in the process according to the invention.

Preferably, a stabilizer is added to the external aqueous phase to stabilize the emulsion/dispersion in order to prevent segregation of the internal non-aqueous (oily) phase and the external aqueous phase.

The preferred stabilizers for producing the polysaccharide- and protein-based microcapsules according to the present invention are especially acrylic copolymers which have sulfonate groups. Also suitable are copolymers of acrylamides and acrylic acid, copolymers of alkyl acrylates and N-vinylpyrrolidone such as LUVISKOL® K15, K30 or K90 (BASF); sodium polycarboxylates, sodium polystyrene sulfonates, vinyl and methyl vinyl ether maleic anhydride copolymers as well as ethylene, isobutylene or styrene maleic anhydride copolymers, microcrystalline cellulose, which is marketed under the name VIVAPUR®, for example, diutane gum, xanthan gum or carboxymethyl celluloses.

The amount of stabilizers used can be in the range of 0.01 to 10% by weight and preferably in the range of 0.1 to 3% by weight, in each case based on the external aqueous phase.

Optionally, an emulsifier, preferably an O/W emulsifier, is used in the process according to the invention, which enables a homogeneous distribution of the oil droplets of the internal non-aqueous phase in the external aqueous phase and stabilizes the emulsion. The same applies to the mixing of solid, non-soluble active ingredients in the external aqueous phase in order to stabilize the resulting dispersion.

The addition of an emulsifier is optional, in particular if the protein or the protective colloid has no or only low, i.e. insufficient, emulsifying properties. If an emulsifying protein and/or protective colloid is used, the use of an emulsifier can advantageously be dispensed with in the process according to the invention.

Suitable emulsifiers include non-ionic surfactants from at least one of the following groups:
addition products of 2 to 30 mol ethylene oxide and/or 0 to 5 mol propylene oxide to linear fatty alcohols with 8 to 22 C— atoms, to fatty acids with 12 to 22 C— atoms, to alkylphenols with 8 to 15 C— atoms in the alkyl group and alkylamines with 8 to 22 carbon atoms in the alkyl radical;
alkyl and/or alkenyl oligoglycosides with 8 to 22 carbon atoms in the alk(en)yl radical and their ethoxylated analogs;
addition products of 1 to 15 moles of ethylene oxide on castor oil and/or hardened castor oil;
addition products of 15 to 60 moles of ethylene oxide on castor oil and/or hardened castor oil;
partial esters of glycerol and/or sorbitan with unsaturated, linear or saturated, branched fatty acids with 12 to 22 carbon atoms and/or hydroxycarboxylic acids with 3 to 18 carbon atoms and their adducts with 1 to 30 moles of ethylene oxide;
partial esters of polyglycerol (average degree of self-condensation 2 to 8), polyethylene glycol (molecular weight 400 to 5000), trimethylolpropane, pentaerythritol, sugar alcohols (e.g. sorbitol), alkyl glucosides (e.g. methyl glucoside, butyl glucoside, lauryl glucoside) and polyglucosides (e.g. cellulose) with saturated and/or unsaturated, linear or branched fatty acids with 12 to 22 carbon atoms and/or hydroxycarboxylic acids with 3 to 18 carbon atoms as well as their adducts with 1 to 30 moles of ethylene oxide, preferably Cremophor®;
mixed esters of pentaerythritol, fatty acids, citric acid and fatty alcohol and/or mixed esters of fatty acids with 6 to 22 carbon atoms, methyl glucose and polyols, preferably glycerol or polyglycerol;
mono-, di- and trialkylphosphates as well as mono-, di- and/or tri-PEG-alkylphosphates and their salts;
lanolin alcohols;
polysiloxane polyalkyl polyether copolymers or corresponding derivatives;
block copolymers, e.g. polyethylene glycol-30 dipolyhydroxystearates;
polymer emulsifiers, e.g. pemulene types (TR-1, TR-2) from Goodrich or Cosmedia® SP from Cognis;
polyalkylene glycols and glycerol carbonate.

Typical anionic emulsifiers that can be used in the process according to the invention for producing the isocyanate-based microcapsules are aliphatic fatty acids with 12 to 22 carbon atoms, such as palmitic acid, stearic acid or behenic acid, and dicarboxylic acids with 12 to 22 carbon atoms, such as azelaic acid or sebacic acid.

Furthermore, zwitterionic surfactants can be used as emulsifiers in the process according to the invention for producing the polysaccharide- and protein-based microcapsules.

Zwitterionic surfactants are those surface-active compounds which carry at least one quaternary ammonium group and at least one carboxylate and one sulfonate group in the molecule. Particularly suitable zwitterionic surfactants are the so-called betaines such as the N-alkyl-N,N-dimethylammonium glycinates, for example cocoalkyldimethylammonium glycinate, N-acylaminopropyl-N,N-dimethylammonium glycinates, for example the cocosacylaminopropyldimethyl ammonium glycinate, and 2-alkyl-3-carboxylmethyl-3-hydroxyethylimidazolines, each having 8 to 18 C— atoms in the alkyl or acyl group, and the cocosacylaminoethylhydroxyethylcarboxymethyl glycinate. Particularly preferred is the fatty acid amide derivative known under the CTFA designation cocamidopropyl betaine.

Ampholytic surfactants are also suitable emulsifiers. Ampholytic surfactants are understood to be those surface-active compounds which, in addition to a C8/18 alkyl or acyl group in the molecule, contain at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyl-taurines, N-alkylsarcosines, 2-alkylaminopropionic acids and alkylaminoacetic acids, each having about 8 to 18 C— atoms in the alkyl group. Particularly preferred ampholytic surfactants are N-cocoalkylaminopropionate, cocoacylaminoethylaminopropionate and C12/18 acylsarcosine.

Finally, cationic surfactants can also be considered as emulsifiers, whereby those of the esterquat type, preferably methyl-quaternized difatty acid triethanolamine ester salts, quaternized hydroxyethyl cellulose, modified chitosan with propylene glycol and quaternized with epichlorohydrin, distearyldimethylammonium chloride (DSDMAC), benzalkonium chloride, benzethonium chloride, cetylalkonium chloride, cetylpyridinium chloride, cetyltrimethylammonium bromide (cetrimonium bromide), dequalinium chloride are particularly preferred.

The emulsifiers can be added to the external aqueous phase in an amount of about 0.1 to about 10 wt.- % and preferably about 1 to about 5 wt.- %, in each case based on the total weight of the external aqueous phase.

Emulsion formation (for liquid active ingredients) or dispersion formation (for solid active ingredients), i.e. the emulsification or dispersion of the internal non-aqueous or oily phase with the external aqueous or hydrophilic phase, takes place under high turbulence or high shear, whereby the strength of the turbulence or shear determines the diameter of the microcapsules obtained. The microcapsules can be produced continuously or discontinuously. As the viscosity of the aqueous phase increases or the viscosity of the oily phase decreases, the size of the capsules generally decreases.

The process according to the invention for producing the polysaccharide- and protein-based microcapsules can, for example, be carried out using the “inline” technique. In this process, the internal non-aqueous phase and the external aqueous phase are initially fed separately to an emulsifying turbine by means of a forced metering pump, combined shortly before entering the emulsifying turbine or combined in the emulsifying turbine, at a throughput volume of 1200 to 1500 I/h. In addition, the process according to the invention for producing the polysaccharide- and protein-based microcapsules can also be carried out in conventional dispersion apparatus or emulsifying devices.

The emulsification or dispersion of the external aqueous phase and the internal non-aqueous phase is carried out for the production of the microcapsules according to the invention, for example by means of an emulsifying turbine (IKA Eurostar 20 high-speed stirrer).

The process of emulsifying or dispersing in the method according to the invention is advantageously carried out for a time of 30 seconds to 20 minutes, preferably from 1 to 7 minutes and most preferably from 1 to 5 minutes, at a stirring speed of 1000 rpm to 5000 rpm, preferably at 2000 rpm to 4000 rpm, until a capsule size of 10 to 75 μm±5 μm (D50) or 75-155±10 μm (D90) has been achieved.

After completion of the emulsifying or dispersing step (iii), an oil-in-water emulsion or dispersion is present, in which the internal oily phase with the active ingredients to be encapsulated is finely emulsified or dispersed in the external aqueous phase in the form of droplets.

In an alternative variant of the process according to the invention, at least one polysaccharide or at least one protein is optionally added in a process step (iv) after the emulsifying or dispersing step (iii), as described above. If the external aqueous phase is provided in process step (ii) only with at least one main protein component, at least one polysaccharide is added in process step (iv). If, on the other hand, the external aqueous phase is provided in process step (ii) only with at least one polysaccharide main component, at least one protein is added in process step (iv). The separate addition results in the formation of multiple layers (“layer-by-layer”), the individual layers of which are crosslinked with one another in the subsequent process step (v). This allows, for example, the charge of the emulsion and thus the aggregation stability to be controlled.

Alternatively, a further protein and/or polysaccharide, which is the same as or different from the at least one protein and/or at least one polysaccharide from process step (ii) or has a different charge or changes its charge when the pH changes, can optionally be added in process step (iv). By adding a further protein and/or polysaccharide, further layers (“layer-by-layer”) are built up, the individual layers of which are crosslinked with one another in the subsequent process step (v). This leads to a denser and more stable network of the capsule wall components and consequently to more stable capsule shells, which increases the stability of the microcapsule.

In a further preferred embodiment of the invention, it may therefore be provided that at least one polysaccharide and/or at least one protein is added in process step (iv).

The further protein and/or further polysaccharide is selected from the group of plant protein and/or polysaccharide as already defined in detail above for process step (ii). The same applies with respect to the preferred variants or preferred combinations of the plant protein and/or polysaccharide described therein.

In a further preferred embodiment of the invention, an external aqueous phase comprising at least one plant protein and at least one first polysaccharide may be provided in step (ii) and at least one polysaccharide and/or at least one protein may additionally be added in step (iv).

Particularly preferably, it can be provided that in step (ii) an external aqueous phase is provided which comprises at least one plant protein and at least one first polysaccharide and that additionally in process step (iv) at least one polysaccharide and/or at least one protein is added, wherein the at least one plant protein in step (ii) and in step (iv) is preferably selected from the group consisting of pea protein, hemp protein, pumpkin protein, soy protein or sunflower protein, wherein the at least one polysaccharide in step (ii) and in step (iv) is a pectin and/or carboxymethylcellulose (CMC).

The pectin is preferably a highly esterified pectin, which preferably has a degree of esterification of at least 50%, particularly preferably a degree of esterification >68%.

In a subsequent process step (v) of the process according to the invention, a first crosslinking of the material of the capsule shell or capsule wall is also carried out with stirring.

The first crosslinking takes place after emulsification or dispersion, optionally by adding a catalyst, in order to crosslink the above-described layers (“layer-by-layer”) of capsule wall components and to stabilize the resulting capsule shell. Catalyzed polymerization reactions between the carboxyl group and/or sulfo group and/or hydroxyl group of polysaccharide and the amino group of protein on the one hand and the isocyanate group of the crosslinking agent on the other hand are formed by interfacial polymerization at the interface between the outer aqueous phase and the dispersed inner phase, i.e. at the interface of the emulsified or dispersed oil droplets which enclose the active ingredient to be encapsulated.

In a further embodiment of the invention, it may thus be provided that in step (v) the first crosslinking is carried out to obtain a microcapsule slurry by adding at least one catalyst; wherein the at least one catalyst is selected from the group consisting of diazobicyclo[2.2.2]octane (DABCO), bismuth catalyst and tin catalyst and mixtures of two or more of the aforementioned catalysts.

Due to the catalyzed crosslinking between the functional groups of the at least one polysaccharide and/or the at least one plant protein of the layers described above and the functional groups of the crosslinking agent, first crosslinking units or a first crosslinking matrix are formed for the construction of a capsule shell or capsule wall.

The formation of the first crosslinking units in the process according to the invention is based on the polyaddition reaction between polysaccharide and crosslinking agent and/or plant protein and crosslinking agent. In this process, the hydroxyl groups of the polysaccharide react with the isocyanate groups of the crosslinking agent to form polyurethane and the amino groups of the protein react with the isocyanate groups of the crosslinking agent to form polyurea. In addition to polyurethane and polyurea, soluble or insoluble complexes of plant protein and polysaccharide are also formed during the first crosslinking step (v) to form the capsule wall matrix or capsule shell.

The higher the number of cross-linking functional groups of the capsule wall building blocks, the greater the spatial cross-linking and the denser and more stable the resulting capsule shell or capsule wall of the microcapsule. In addition to the number of functional groups, the chain length of the individual capsule wall building blocks also significantly influences the mechanical properties, i.e. the stability, of the microcapsules: For example, the large number of hydroxyl groups in starch enables the formation of particularly pronounced spatial cross-links; longer-chain capsule wall building blocks, for example polyisocyanates, lead to the formation of more stable capsule walls.

By forming the first cross-linking matrix or first cross-linking units, the emulsified or dispersed oil droplets with the core material, i.e. the encapsulated active ingredients, are enclosed by the cross-linking matrix or the cross-linking units on the outside at the interface, thus generating a capsule wall, which makes diffusion of the encapsulated active ingredient more difficult.

The addition of the at least one catalyst to the emulsion or dispersion accelerates the crosslinking reaction between the polysaccharide and/or plant protein and the crosslinking agent and catalyzes the reaction in favor of the formation of a first crosslinking matrix or of first crosslinking units.

The catalyst which may be added in the process according to the invention may preferably be diazabicyclo[2.2.2]octane (DABCO), also known as triethylenediamine (TEDA), a bicyclic tertiary amine. DABCO is generally used as a catalyst for the production of polyurethane plastics. The tertiary amine with free electron pairs favors the reaction between the isocyanate groups of the crosslinking agent and the hydroxyl groups of the polysaccharide.

In addition to DABCO, catalysts based on bismuth or tin, for example, such as catalysts based on bismuth(II) salts or bismuth(III) salts, as described in K. C. Frisch & L. P. Rumao, Catalysis in Isocyanate Reactions, Polymer Reviews, 1970, 5:1, pages 103-149, DOI: 10.1080/15583727008085365, whose disclosure in this regard is incorporated in its entirety in the present description, can also be used to catalyze the first crosslinking.

Preferably, a combination of DABCO and one of the above catalysts can be used. Such a mixture leads to a multiplication of reactivity as described in K. C. Frisch & L. P. Rumao, Catalysis in Isocyanate Reactions, Polymer Reviews, 1970, 5:1, pages 103-149, DOI: 10.1080/15583727008085365, the disclosure of which is incorporated in its entirety in the present description.

DABCO and the aforementioned catalysts preferably catalyze the polyurethane reaction between the at least one polymerizable aliphatic polyisocyanate with two or more isocyanate groups and the diols or polyols in the process according to the invention.

In one embodiment of the process according to the invention, the catalyst can be added to the external aqueous phase. It may be provided that the amount in which the catalyst is added to the external aqueous phase is in a range from 0.001 to 2% by weight, preferably in a range from 0.02 to 1.0% by weight and particularly preferably in a range from 0.05 to 0.8% by weight, based on the total weight of the external aqueous phase.

However, larger quantities of catalyst can be added if required.

Preferably, the catalyst is added to the emulsion or dispersion either as such, for example as a solid, or in the form of an aqueous solution, preferably in water, with stirring. It may be provided that the amount of the catalyst is in a range from 0.001 to 2% by weight, preferably in a range from 0.02 to 1.0% by weight and particularly preferably in a range from 0.05 to 0.8% by weight, based on the total weight of the external aqueous phase.

It may preferably be provided that the catalyst is present in the aqueous solution in a concentration of 0.5 to 2 mol/l, preferably 1 mol/l.

The catalyst is added at a stirring speed of 500 rpm to 2000 rpm, preferably at 1000 rpm to 1500 rpm and at a temperature in the range of 20° C. to 30° C., preferably at temperatures of 22° C. to 26° C.

Even more preferably, the process step (v) first crosslinking is carried out by heating the emulsion or dispersion stepwise to a temperature in the range of 60° C. to 90° C., preferably to a temperature in the range of 65 to 85° C., most preferably to a temperature in the range of 70 to 80° C. The first crosslinking in the process according to the invention is carried out for a period of time of about 30 minutes to 90 minutes, preferably for a period of time of 40 minutes to 70 minutes and most preferably for a period of time of 60 minutes.

In a further embodiment of the present process, the first crosslinking can take place at a temperature of 20° C. to 40° C., preferably 20° C. to 30° C., in particular at room temperature.

After the initial cross-linking and formation of the capsule shell or capsule wall, the capsules produced according to the method of the invention are present as raw microcapsules in the form of an aqueous dispersion or a slurry.

After cross-linking, the microcapsules in the slurry still have a flexible shell that is not particularly stable and therefore breaks open easily. For this purpose, the shell is hardened.

The hardening in process step (vi) is preferably carried out by gradually raising the microcapsule slurry to a temperature of at least 60° C., preferably to a temperature in the range from 60° C. to 90° C., preferably in the range from 65° C. to 80° C. up to a maximum of the boiling point of the microcapsule slurry. The curing is usually carried out for a period of at least 1.5 hours, preferably for a period of between 2 and 5 hours, most preferably for a period of 3 hours.

It is also advantageous to add hardening substances to the microcapsule slurry. For this purpose, natural plant tanning agents of the tannin type are used, which from a chemical point of view are proanthocyanidins found in dicotyledonous shrubs, bushes and leaves, particularly in the tropics and subtropics. The terpenes generally have molecular weights in the range of 500 to 3000 KDa. A preferred example of a suitable tannin is corigallin. For hardening, an aqueous preparation of the tannins is added to the aqueous dispersion containing the raw microcapsules. Typically, the tannins are added in amounts of from about 0.1 to about 2 wt.- % and preferably from about 0.5 to about 1.5 wt.- %, based on the microcapsules.

To optimize the cross-linking of the capsule wall matrix, in an alternative variant of the process according to the invention, a further plant protein and/or further polysaccharide can optionally be added to the microcapsule slurry in process step (vi).

The further plant protein and/or further polysaccharide is selected from the group protein and/or polysaccharide as already defined in detail above for process step (ii). The same definitions and preferred embodiments and/or preferred combinations as for the plant protein and/or polysaccharide are also fully valid for the further plant protein and/or polysaccharide.

The further plant protein and/or the further polysaccharide may be the same as or different from the plant protein and/or polysaccharide of process step (ii).

The addition of a further plant protein and/or further polysaccharide leads to further cross-linking with the cross-linking agent and contributes to the formation of a particularly dense and stable network of the capsule wall building blocks.

The curing step (vi) of the method according to the invention is followed by a step of cooling the microcapsule slurry to room temperature and optionally a second crosslinking step of the capsule wall building blocks by adding a further crosslinking agent.

At least one further crosslinking agent selected from the group consisting of transglutaminase, peroxidase, secondary plant substances selected from the group consisting of polyphenols, in particular tannin, gallic acid, ferulic acid, hesperidin, cinnamaldehyde, vanillin, carvacrol, as well as mixtures of two or more of the aforementioned crosslinking agents, as already described above in connection with the first and further crosslinking agents, is used as a further crosslinking agent for further crosslinking in the process according to the invention. The same definitions and preferred embodiments as for the first and further crosslinking agent are also fully valid for the further crosslinking agent.

Cinnamaldehyde, tannin, ferulic acid and gallic acid are particularly preferred among the other crosslinking agents mentioned above.

The at least one further crosslinking agent can be added in the non-aqueous phase. Alternatively or additionally, the at least one further crosslinking agent can be added in the aqueous phase.

The content of further crosslinking agent is in a range of 0.1 to 5 wt.- %, preferably in a range of 0.15 to 2.5 wt. %, based on the total weight of the non-aqueous phase. Most preferably, the crosslinking agent is used in the internal non-aqueous phase in a range of 0.1 to 1% by weight, based on the total weight of the non-aqueous phase.

The further crosslinking agent can be added to the emulsion or dispersion either as such, for example as a solid, or in the form of an aqueous solution.

Even more preferably, the further crosslinking in process step (vii) is carried out by heating the emulsion or dispersion stepwise to a temperature in the range from 20° C. to 50° C., preferably to a temperature in the range from 30° C. to 40° C. The further crosslinking in the process according to the invention is carried out for a period of time of about 20 minutes to 10 hours, preferably for a period of time of 30 minutes to 8 hours.

To optimize the first crosslinking in process step (v) and/or the further crosslinking in process step (vii) of the process according to the invention, the pH value of the emulsion or dispersion is optionally adjusted to a pH value above or below the isoelectric point of the protein used. At a pH value below the isoelectric point, the net electrostatic charge of a protein is positive; above the isoelectric point, the net charge of a protein is negative.

Preferably, the pH is adjusted to a pH in the range of pH 2.0 to pH 4.0, even more preferably to a pH in the range of pH 2.5 to pH 3.5 and most preferably to a pH in the range of pH 2.9 to pH 3.3 to obtain a positive charge of the protein. In order to obtain a negative charge of the protein, the pH is preferably adjusted to a pH in the range of pH 8.0 to pH 12.0, even more preferably to a pH in the range of pH 9.0 to pH 10.0 and most preferably to a pH in the range of 9.3 to 9.6.

For this purpose, an organic acid, for example formic acid or acetic acid, or a base, for example caustic soda, is added to the emulsion or dispersion and a pH value in the above-mentioned ranges is set.

Carrying out the first cross-linking and/or further cross-linking at a pH value above or below the isoelectric point has the advantage that the electrical charge of the protein is changed and thus electrostatic interactions can positively influence capsule formation. In addition, such a modification of the proteins has a positive effect on their emulsifying capacity.

During the first and any further crosslinking steps, the stirring power is reduced, for example to a stirring speed of around 800 to 1400 rpm, in order to prevent the microcapsules from breaking up again immediately.

An important criterion for the usability of microcapsules is the weight ratio of core material to capsule wall material. While on the one hand the aim is to achieve the highest possible proportion of core material in order to maximize the utility value of the capsules, on the other hand it is necessary for the capsule to still have a sufficient proportion of capsule wall material to ensure the stability of the capsules.

According to the invention, it has proven to be particularly advantageous that the microcapsules are designed in such a way that the microcapsules have a weight ratio of core material to capsule wall material of 50:50 to 98:2, preferably 80:20 to 97.5:2.5.

After complete curing, the microcapsules produced by the process according to the invention are available as a dispersion in water, which is also known as a microcapsule dispersion or microcapsule slurry. In this form, the microcapsules are basically ready for sale.

In order to prevent segregation or creaming of such a suspension and thus achieve high storage stability, it has proven advantageous for the suspension to have a viscosity of 12 to 2000 mPas. To obtain the desired viscosity of the suspension, a thickening agent is preferably used.

Xanthan gum, diuthan gum, carboxymethyl cellulose (CMC), microcrystalline cellulose (MCC) or guar gum are preferably used as thickening agents.

To improve the shelf life, one or more preservatives can optionally be added to the microcapsule slurry or the microcapsule slurry can be dried.

The preservatives preferably used are 1,2-hexanediol, 1,2-octanediol, phenoxyethanol-based products, products from mixtures of 1,2-benzisothia-zolin-3-one (2.5%) and 2-methyl-4-isothiazolin-3-one (2.5%), sodium benzoate or the like.

Alternatively, and for preservation purposes, the microcapsule slurry is preferably dried.

Processes such as lyophilization can be used to dry the microcapsule slurry, but spray drying, for example in a fluidized bed, is preferred. It has proven to be advantageous to add further polysaccharides, preferably dextrins and in particular maltodextrins, to the suspension at temperatures of around 20 to around 50° C. and preferably around 40° C., which support the drying process and protect the capsules during this process. The amount of polysaccharides used can be about 50 to about 150% by weight and preferably about 80 to about 120% by weight relative to the capsule mass in the dispersion.

The spray drying itself can be carried out continuously or in batches in conventional spray systems, with the inlet temperature being about 170 to about 200° C. and preferably about 180 to 185° C. and the outlet temperature being about 70 to about 80° C. and preferably about 72 to 78° C.

The catalyzed crosslinking of polysaccharide and/or protein with the crosslinking agent and optionally further crosslinking agent introduces large molecules into the network of the capsule shell, which increase the proportion of natural components in the capsule shell or in the microcapsule slurry and which thus increase the biodegradability of the capsule shell, as illustrated in the following embodiment examples.

The process according to the invention is further characterized by the fact that plant protein, polysaccharide and aliphatic polyisocyanate(s) are polymerized and/or crosslinked as the main components, thus enabling the production of very stable vegan microcapsules with excellent sensory properties based on biocompatible polymers. Another particularly advantageous feature of the process according to the invention is that the polyisocyanates no longer act as the main material in the plant protein-based microcapsules according to the invention, but instead serve predominantly as crosslinking agents for the amino acids and the other above-mentioned components. This reduction in the quantities of polyisocyanate to be used is characterized as a further advantage of the process according to the invention.

The process according to the invention thus makes it possible to replace part of the polyisocyanate with biodegradable wall materials such as proteins and/or polysaccharides, thereby reducing the polyisocyanate content without any loss or reduction in the functionality of the microcapsules, such as olfactory properties and positive secondary properties such as high stability, namely the ability to retain the active ingredient. The process according to the invention can thus be used to produce microcapsules which, on the one hand, have excellent functionality and, at the same time, are readily biodegradable.

In order to obtain plant protein-based microcapsules which have outstanding stability properties and which also have excellent sensory properties and, moreover, make it possible to reduce the amount of aliphatic polyisocyanates used, it may be provided in a highly preferred embodiment of the process according to the invention that the polysaccharide or the at least one further polysaccharide is hyaluronic acid or wherein the polyhydroxyphenol is a tannin.

It may be possible here that the polysaccharide is the first polysaccharide according to step (ii). Alternatively or additionally, it may be possible that the polysaccharide is the further polysaccharide according to step (iv) and/or step (vi).

Surprisingly, it has been found that it can be particularly advantageous if the amount of hyaluronic acid relative to the amount of wall former is 1 to 15 percent by weight, preferably 2 to 13 percent by weight, particularly preferably 2.5 to 6 percent by weight.

It is particularly preferable for hyaluronic acid to be added after the emulsion has formed.

In particular, it may also be provided that the content of hyaluronic acid is in a range from 0.1 to 5-% by weight, preferably in a range from 0.15 to 2.5% by weight, particularly preferably in a range from 0.2 to 1.0% by weight % based on the total weight of the non-aqueous internal phase and/or in a range from 0.1 to 5% by weight-, preferably in a range from 0.15 to 2.5% by weight, particularly preferably in a range from 0.25 to 1.0% by weight based on the total weight of the aqueous external phase.

On the one hand, this can reduce the amount of aliphatic polyisocyanates used for crosslinking, which can have a positive impact on the environment. On the other hand, the reduction in the quantities of polyisocyanates can have a positive effect on the biodegradability of the plant protein-based microcapsules. Another advantage is that neither the stability nor the sensory properties of the microcapsules are negatively affected.

In a further preferred embodiment of the process according to the invention, it may be provided that, in addition to the addition of hyaluronic acid, at least one catalyst is added after the emulsion has formed.

This makes it possible to obtain particularly stable plant protein-based microcapsules with very good sensory properties (odor or fragrance release) in total reaction times of less than 7 hours.

In a second aspect, the present invention relates to a plant protein-based microcapsule or a corresponding microcapsule slurry prepared according to the method of the invention.

The plant protein-based microcapsules according to the invention are characterized in that they consist of or comprise:
(a) a core comprising or consisting of at least one hydrophobic active ingredient;
(b) a capsule shell comprising or consisting of a crosslinking matrix or crosslinking units of at least one plant protein and at least one aliphatic polyisocyanate as crosslinking agent and optionally at least one polysaccharide; as well as optionally at least one protective colloid and/or optionally at least one further crosslinking agent.

The microcapsule according to the invention comprises a core which is surrounded or encased by the capsule shell or capsule wall. Any material that is suitable for inclusion in microcapsules can be used as the core material for producing the microcapsules according to the invention. The materials to be encapsulated are preferably hydrophobic, water-insoluble or water-immiscible liquids or solids as well as suspensions.

In the context of the present description, the core material is a hydrophobic active ingredient, i.e. a substance that has a specific effect or causes a specific reaction, for example a drug, a plant protection agent, a cosmetic active ingredient, a food active ingredient, etc., as described above. The term “hydrophobic active ingredient” means that the active ingredient to be encapsulated is in the internal non-aqueous phase during the production of the microcapsule and does not mix with the external aqueous phase.

The polymerization and/or crosslinking of functional groups of plant protein and/or polysaccharide with polyisocyanate results in a stable capsule wall of alternating and dense and thus stable crosslinking matrices or crosslinking units based on polyurea and polyurethane as well as soluble or insoluble complexes of protein and polysaccharide.

In a preferred embodiment, the capsule shell comprises or consists of: a crosslinking matrix or crosslinking units comprising a polymerization and/or crosslinking of at least one plant protein with the first and optionally the further crosslinking agent and/or a crosslinking matrix or crosslinking units comprising a polymerization and/or crosslinking of at least one polysaccharide, with the first and optionally the further crosslinking agent.

The crosslinking matrix or the crosslinking units from a polymerization and/or crosslinking of at least one plant protein with the first and optionally the further crosslinking agent is predominantly a polyurea-based network and the crosslinking matrix or the crosslinking units from a polymerization and/or crosslinking of at least one polysaccharide with the first and optionally the further crosslinking agent is predominantly a polyurethane-based network as well as soluble or insoluble complexes of plant protein and polysaccharide.

In addition to the polyurea formation and/or polyurethane formation described above, the crosslinking steps described above produce by-products such as urea, allophanate, biuret, uretidione, carbodiimide, uretonimine, etc. due to the reactivity of the polyisocyanates, as described in M. F. Sonnenschein, Introduction to Polyurethane Chemistry, Polyurethanes: Science, Technology, Markets, and Trends, First Edition, 2015, John Wiley & Sons, pages 105 to 126, the disclosure of which is incorporated herein by reference in its entirety. These by-products are part of the capsule shell or capsule wall.

The structure of the capsule wall, based on several individual defined and alternating crosslinking matrices or crosslinking units, makes it possible to produce particularly stable microcapsules with outstanding sensory performance, while at the same time significantly reducing the number of shell components.

In addition to the main components listed above, the capsule shell may optionally comprise a protective colloid and/or optionally a further cross-linking agent.

It is particularly advantageous if the plant protein-based microcapsule comprises hyaluronic acid. Surprisingly, it has been found that this can significantly improve the stability properties of the plant protein-based microcapsule. In addition, such plant protein-based microcapsules surprisingly exhibit improved sensory properties. It was also found that the amount of polyisocyanates used in the production of these plant protein-based microcapsules comprising hyaluronic acid can be reduced.

In a preferred variant according to the second aspect, the microcapsules according to the invention are in the form of a dispersion or slurry in which the microcapsules are dispersed in the external aqueous phase. The proportion by weight of the microcapsules in the dispersion or slurry is about 20 to 60 wt.-%, in particular about 25 to 55 wt.-%, more preferably about 33 to 50 wt.-%.

Surprisingly, the plant protein-based microcapsule produced according to the method of the invention has a comparable stability and a comparable content of unintentionally leaking perfume oil as microcapsules of the prior art, despite a reduction of the polyisocyanate content in the microcapsule wall.

Due to the excellent stability and an excellent release capacity of the microcapsules and the possibility of encapsulating a broad spectrum of hydrophobic active ingredients with the microcapsules according to the invention, the plant protein-based microcapsules according to the present invention can be used for a wide range of applications for scenting and flavoring.

Furthermore, the microcapsule according to the invention is a universal capsule which can be used to encapsulate a broad spectrum of fragrances or flavorings, even fragrances or flavorings which have an aldehyde, carboxylic acid or ester functionality, so that there are no restrictions against individual active ingredients.

Due to their advantageous properties, in particular their stability and the targeted release of the active ingredients, the plant protein-based microcapsules according to the invention are suitable for a wide range of applications and in particular for use in household products, textile care products, detergents, fabric softeners, cleaning agents, scent boosters, scent lotions and fragrance enhancers, cosmetics, personal care products, agricultural products, pharmaceutical products, or print coatings for paper and the like.

The present invention therefore relates in a further aspect to the use of the plant protein-based microcapsules according to the invention or a dispersion of the plant protein-based microcapsules according to the invention (microcapsule slurry) for the manufacture of household products, textile care products, detergents, fabric softeners, cleaning agents, scent boosters, scent lotions and fragrance enhancers in liquid or solid form, cosmetics, personal care products, agricultural products, pharmaceutical products or print coating for paper. The microcapsules according to the invention are particularly suitable for the inclusion of hydrophobic fragrances or flavorings, which can be used in various household and textile care products.

Finally, the present invention relates to household products, fabric care products, laundry detergents, fabric softeners, cleaning products, scent boosters, scent lotions and fragrance enhancers, cosmetics, personal care products, agricultural products, pharmaceutical products or printing coating for paper and the like comprising the plant protein-based microcapsules according to the invention or a dispersion of the plant protein-based microcapsules according to the invention.

The proportion of microcapsules in the aforementioned products is 0.05 to 15% by weight, based on the total weight of the product, preferably 0.2 to 5% by weight.

The invention further relates to a microcapsule slurry comprising a microcapsule according to the invention in combination with a thickener and/or a preservative.

### EMBODIMENT EXAMPLES

The plant protein-based microcapsules according to the invention and their advantageous properties are described in more detail with reference to the following examples.

The following stability data refer to a test at 40° C. in a commercially available formulation such as Scent Booster or fabric softener.

Vegan microcapsules according to the invention were produced with various plant proteins, namely pea protein, hemp protein, soy protein, sunflower protein and pumpkin protein. The production with pea protein is explained below as an example.

The pea protein can be Pea protein Nutralys® F858F (Roquette). The soy protein may or may not be Soya Protein Isolate SolPro 921 (Solbar Ningbo Technology Co., Ltd.). The hemp protein may or may not be Hemp Protein 50 (All Organic Treasures GmbH). The pumpkin protein may or may not be Pumpkin Protein 60 (All Organic Treasures GmbH).

The non-aqueous internal phase comprises a polyisocyanate mixture consisting of TAKENATE™ D-120N, STABIO D370N and Desmodur N 3400. These three polyisocyanates were each used in equal proportions. In addition to the pea protein, the aqueous external phase comprises tannin, CMC and glycerol (86.5% in water). The pH of the aqueous external phase was adjusted to 3.3 using formic acid. After emulsion formation, hyaluronic acid, maltodextrin and DABCO were added. In the final step, curing took place at 70° C. over a period of 3 hours.

Particularly stable microcapsules can be obtained if curing takes place at a temperature between 70° C. and 80° C. over a period of 2 hours to 4 hours.

In particular, it may also be possible to cure for 2 hours at 80° C. in order to obtain particularly stable microcapsules with very good sensory properties.

The amount of free oil was measured in isopropanol, i.e. a defined amount of the microcapsule slurry was mixed with isopropanol, stirred for 30 seconds and a sample was taken. The sample taken was measured using GC-MS. The result indicates how much of the encapsulated oil was transferred to the isopropanol or was not completely encapsulated. The free oil content therefore provides an indication of whether the process itself has worked, i.e. whether the perfume oil has been completely encapsulated and/or whether the capsule shell is stable enough to prevent the perfume oil from bleeding into isopropanol. Values of less than 1% are considered an indicator of successful encapsulation and a stable capsule shell.

### Example 1: Stability of the Plant Protein-Based Microcapsules According to the Invention in Use

The stability of microcapsules according to the invention and microcapsules according to the prior art, which were produced as described above or analogously, was measured in the target application. The stability test was carried out using a representative softener into which the microcapsule slurry was incorporated in an amount of 1% by weight and stored at room temperature and at 40° C. in each case. Samples were taken at defined intervals and the stability measured. The identification of the fragrance components is based on an in-house database and commercially available analytical databases of fragrance formulas. This results in a percentage of the perfume oil remaining in the capsule. A result of 98%, for example, means that 2% of the amount of perfume oil originally used is no longer in the capsule.

In the tables and figures shown below, the common abbreviations “d” for “day” and “w” for “week” have been used.

Table 1 below shows that the plant protein-based microcapsules according to the invention, which were produced using only aliphatic polyisocyanates as crosslinking agents, have significantly better stability than plant protein-based microcapsules in which aromatic and aliphatic polyisocyanates were used.

TABLE 1

| | Stability in fabric softener | | | |
|---|---|---|---|---|
| | @ 40° C. [%] | | | |
| Plant protein | 1 d | 1 w | 2 w | 4 w |
| Pea protein, aliphatic isocyanates | 92 | 85 | 80 | 79 |
| Pea protein, mix of aromatic and aliphatic | 73 | 59 | 52 | 44 |
| isocyanates | | | | |
| Sunflower protein, aliphatic isocyanates | 80 | 75 | 74 | 69 |
| Sunflower protein, mix | 55 | 47 | 40 | 34 |
| of aromatic and aliphatic | | | | |
| isocyanates | | | | |
| Hemp protein, aliphatic isocyanates | 95 | 86 | 79 | 74 |
| Hemp protein, mix of | 57 | 45 | 39 | 33 |
| aromatic and aliphatic | | | | |
| isocyanates | | | | |
| Pumpkin protein, aliphatic isocyanates | 92 | 80 | 74 | 67 |
| Pumpkin protein, mix of | 7 | 1 | — | — |
| aromatic and aliphatic | | | | |
| isocyanates | | | | |
| Gelatine, aliphatic isocyanates | 61 | 36 | 32 | 30 |
| Gelatine, mix of aromatic and aliphatic | 39 | 19 | 15 | 9 |
| isocyanates | | | | |

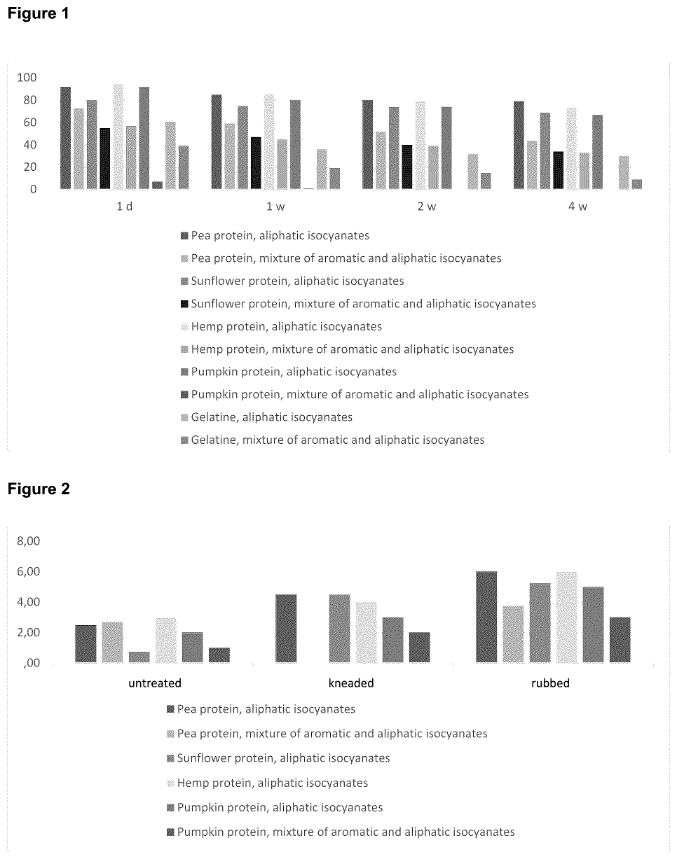

The results according to Table 1 are also shown in FIG. 1.

In addition, it can be seen from the stability data and comparisons shown in Table 1 that, without exception, all the plant proteins used, i.e. pea protein, sunflower protein, pumpkin protein and hemp protein, lead to plant protein-based microcapsules according to the invention which have very good stability properties. This is not the case when animal proteins are used, as can be shown using the example of gelatine. The stability of gelatine-containing microcapsules is reduced compared to the microcapsules according to the invention when aliphatic polyisocyanates are used exclusively.

TABLE 2

| Polyisocyanate | Polyisocyanate | Polyisocyanate | Polyisocyanate | Stability in fabric softener at 40° C. [%] | | | |
|---|---|---|---|---|---|---|---|
| 1 | 2 | 3 | 4 | 1 d | 1 w | 2 w | 4 w |
| 100% | | | | 98 | 97 | 96 | 94 |
| | 100% | | | 73 | 57 | 47 | 38 |
| | | 100% | | 40 | 22 | 16 | 13 |
| | | | 100% | 0 | 2 | — | — |

In Table 2 above, “Polyisocyanate 1” is TAKENATE™ D-120N. “Polyisocyanate 2” is STABIO D370N. “Polyisocyanate 3” is Desmodur N 3400 and “Polyisocyanate 4” is 100741 Desmodur 44 M Flakes. As is known, the latter is an aromatic polyisocyanate. The shown stabilities all refer to pea protein-based ones, which were obtained as described above.

From the data in Table 2, it can be deduced that plant protein-based microcapsules, which have extremely poor stabilities when only one and exclusively aromatic polyisocyanate is used as crosslinker (polyisocyanate 4). In contrast, significantly improved stabilities of the plant protein-based microcapsules according to the invention can be achieved if aliphatic polyisocyanates (polyisocyanate 2 or polyisocyanate 3) are used. Surprisingly, the best results in terms of stability can be achieved when a cycloaliphatic polyisocyanate (polyisocyanate 1) is used as a crosslinking agent for the production of plant protein-based microcapsules. This is particularly true with regard to long-term stability, i.e. stability over a period of 4 weeks.

Further investigations into the stability of the plant protein-based microcapsules according to the invention were also carried out with regard to the use of polyisocyanate mixtures as crosslinking agents. The data and results for this are shown in Table 3 below. By analogy with the above explanations, the “polyisocyanate 1” named in Table 3 is TAKENATE™ D-120N. “Polyisocyanate 2” is STABIO D370N. “Polyisocyanate 3” is Desmodur N 3400 and “Polyisocyanate 4” is Desmodur 44 M Flakes. The stabilities shown all relate to pea protein-based microcapsules.

TABLE 3

| Polyisocyanate | Polyisocyanate | Polyisocyanate | Polyisocyanate | Stability in fabric softener at 40° C. [%] | | | |
|---|---|---|---|---|---|---|---|
| 1 | 2 | 3 | 4 | 1 d | 1 w | 2 w | 4 w |
| 50% | 50% | | | 94 | 88 | 86 | 84 |
| 50% | | 50% | | 82 | 72 | 70 | 62 |
| | 50% | 50% | | 60 | 36 | 19 | 17 |
| | 50% | | 50% | 55 | 38 | 31 | 24 |
| 75% | 25% | | | 95 | 92 | 89 | 89 |
| 75% | | 25% | | 92 | 87 | 85 | 84 |
| | 75% | 25% | | 64 | 43 | 27 | 21 |
| 25% | | | 75% | 51 | 34 | 26 | 19 |

Surprisingly, it was found in the context of the present invention that very good stabilities of the plant protein-based microcapsules according to the invention can also be obtained when (as shown above in Table 2) only aliphatic isocyanates are used. The stability decreases accordingly as soon as an aromatic isocyanate is used even proportionally or partially. It is therefore essential to the invention that at least one aliphatic polyisocyanate is used as the crosslinking agent in step (i) and, in particular, that no aromatic polyisocyanates are used.

Furthermore, it was surprisingly found that particularly good stabilities of the plant protein-based microcapsules according to the invention are obtained when two aliphatic isocyanates, in particular a cycloaliphatic (polyisocyanate 1) and an aliphatic (polyisocyanate 2 or polyisocyanate 3) polyisocyanate are used.

Due to the fact that the sensory performance, i.e. the release of fragrance or odorant, is less good with polyisocyanate 1, very good stability can be achieved with simultaneously very good sensory properties when using a mixture of two or more polyisocyanates as crosslinking agents, one of which is a cycloaliphatic and one of which is an aliphatic polyisocyanate. Thus, in view of the two factors of stability and sensory properties, the use of two or more polyisocyanates as crosslinking agents in the production of plant protein-based microcapsules is preferred. A further advantage of the use of two or more polyisocyanates as crosslinking agents compared with only one aliphatic crosslinking agent is that the plant protein-based microcapsules produced in this way according to the invention are more biodegradable and, moreover, smaller quantities of polyisocyanates have to be used overall.

The use of three polyisocyanates, as shown in Table 4 below, can also lead to very stable plant protein-based microcapsules according to the present invention.

When using a mixture consisting of three polyisocyanates, particularly good stability is achieved if at least one cycloaliphatic polyisocyanate is present. For improved sensory properties and biodegradability, at least one aliphatic polyisocyanate is present.

With ratios of the three polyisocyanates of 60:20:20, as shown in Table 4, improved sensory properties and improved biodegradability can be obtained in particular if a cycloaliphatic polyisocyanate and an aliphatic polyisocyanate are each used in equal amounts and a further aliphatic polyisocyanate is used in three times the amount of the cycloaliphatic polyisocyanate (thus a ratio of 20:60:20, see last column of Table 4).

Within the scope of the invention, molar ratios given herein may also be replaced by quantitative ratios and vice versa.

Surprisingly, very good results in terms of stability and sensory properties are achieved when three polyisocyanates are used in equal proportions, based on the respective quantities.

TABLE 4

| Polyisocyanate | Polyisocyanate | Polyisocyanate | Polyisocyanate | Stability in fabric softener at 40° C. [%] | | | |
|---|---|---|---|---|---|---|---|
| 1 | 2 | 3 | 4 | 1 d | 1 w | 2 w | 4 w |
| 33% | 33% | 33% | | 92 | 85 | 80 | 79 |
| 33% | | 33% | 33% | 79 | 68 | 63 | 60 |
| | 33% | 33% | 33% | 72 | 66 | 58 | 50 |
| 60% | 20% | 20% | | 95 | 91 | 91 | 88 |
| 20% | 60% | 20% | | 88 | 83 | 79 | 74 |

In analogy to the above explanations, the “polyisocyanate 1” named in Table 4 is TAKENATE™ D-120N. For “Polyisocyanate 2” STABIO D370N. “Polyisocyanate 3” is Desmodur N 3400 and “Polyisocyanate 4” is 100741 Desmodur 44 M Flakes. The stabilities shown all relate to pea protein-based microcapsules.

The developers have also discovered that the stability of the plant protein-based microcapsules according to the invention can also be positively influenced by the addition of other substances. Surprisingly, it was found that the addition of hyaluronic acid, in particular the addition of hyaluronic acid after the emulsification step, has a significant positive effect on the stability of the microcapsules produced, as shown in Table 5 below. In Table 5, the percentages of isocyanates refer to the total amount of wall former in the plant protein-based microcapsule.

TABLE 5

| | Stability in fabric softener | | | |
|---|---|---|---|---|
| | @ 40° C. [%] | | | |
| Capsule type | 1 d | 1 w | 2 w | 4 w |
| Pea protein, 28% isocyanates | 79 | 60 | 53 | 49 |
| Pea protein, 28% isocyanates | 88 | 80 | — | 70 |
| with hyaluronic | | | | |
| acid | | | | |
| Pea protein, 23% isocyanates | 77 | 60 | 52 | 48 |
| Pea protein, 23% isocyanates | 88 | 76 | — | 64 |
| with hyaluronic | | | | |
| acid | | | | |
| Pea protein, 19% isocyanates | 55 | 44 | — | 38 |
| Pea protein, 19% isocyanates | 84 | 69 | — | 57 |
| with hyaluronic | | | | |
| acid | | | | |
| Gelatine, 28% isocyanates | 61 | 36 | 32 | 30 |
| Gelatine, 23% isocyanates | 60 | 46 | 37 | 31 |

The addition of hyaluronic acid therefore has a positive effect on stability, whereby remarkably fewer polyisocyanates are required as crosslinking agents. Thus, the use of hyaluronic acid in the process according to the invention for the production of plant protein-based microcapsules is also advantageous in that the microcapsules obtained have improved biodegradability. In addition, the use of hyaluronic acid improves the sensory properties.

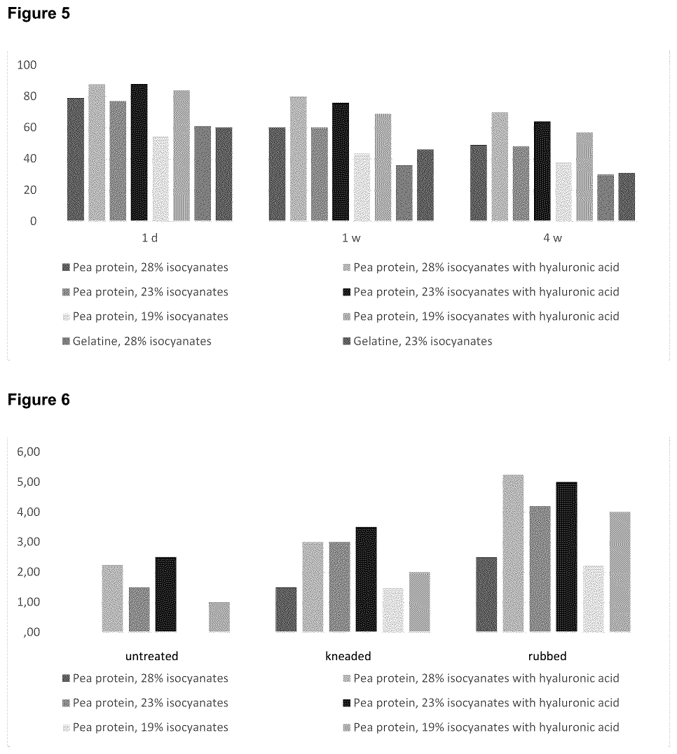

The results according to Table 5 are also shown in FIG. 5.

Results of further studies on the stability behavior of plant protein-based microcapsules according to the invention are shown in Table 6.

TABLE 6

| | Stability in fabric softener | | | |
|---|---|---|---|---|
| | @ 40° C. [%] | | | |
| Capsule type | 1 d | 1 w | 2 w | 4 w |
| Pea protein, 28% isocyanates | 84 | 71 | 64 | 56 |
| Pea protein, 23% isocyanates | 76 | 63 | 55 | 46 |
| Pea protein, 19% isocyanates | 75 | 56 | 54 | 45 |
| Pea protein, 28% isocyanates | 88 | 80 | — | 70 |
| with glycerine and | | | | |
| hyaluronic acid | | | | |
| Pea protein, 23% isocyanates | 88 | 76 | — | 64 |
| with glycerine and | | | | |
| hyaluronic acid | | | | |
| Pea protein, 19% isocyanates | 84 | 69 | — | 57 |
| with glycerine and | | | | |
| hyaluronic acid | | | | |

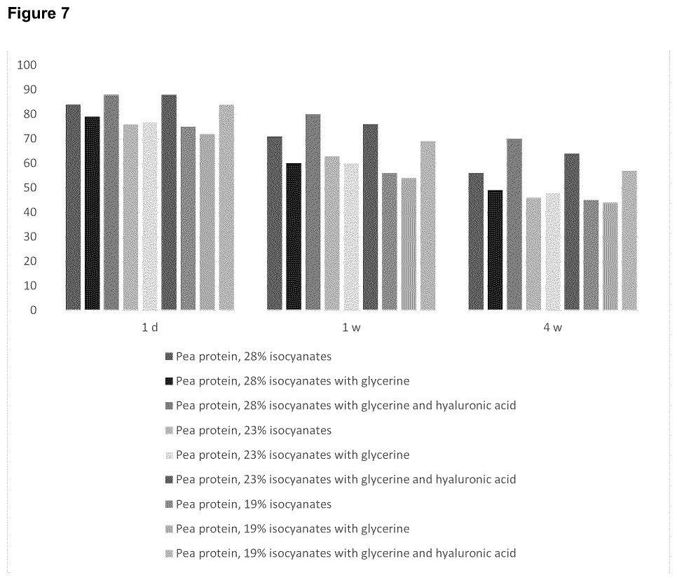

The results according to Table 6 are also shown in FIG. 7.

It was surprisingly found that the further addition of glycerol to pea protein-based microcapsules that already contain hyaluronic acid (see above) also has a positive effect on the stability of the microcapsules according to the invention. What is particularly advantageous about the addition of both glycerol and hyaluronic acid is that the quantities of polyisocyanate can be reduced. Biodegradability increases when the amount of isocyanate (crosslinker) decreases.

With regard to the plant proteins used, pea protein, sunflower protein, hemp protein and pumpkin protein are particularly preferred in the context of the present invention in terms of the stability of the microcapsules obtained in each case. Correspondingly good stabilities of these four preferred plant proteins were achieved compared to other plant proteins. High stabilities were also achieved with soy protein and fava bean protein, as can be seen from Table 7 shown below and FIG. 4.

TABLE 7

| | Stability in fabric softener @ | | | |
|---|---|---|---|---|
| | 40° C. [%] | | | |
| Capsule type | 1 d | 1 w | 2 w | 4 w |
| Pea protein, aliphatic isocyanates | 92 | 85 | 80 | 79 |
| Sunflower protein, aliphatic isocyanates | 80 | 75 | 74 | 69 |
| Hemp protein, aliphatic isocyanates | 95 | 86 | 79 | 74 |
| Pumpkin protein, aliphatic isocyanates | 92 | 80 | 74 | 67 |
| Soy protein, aliphatic isocyanates | 65 | 64 | 54 | 46 |
| Fava bean protein, aliphatic isocyanates | 62 | 54 | 47 | 39 |

### Example 2: Sensory Evaluation of the Plant Protein-Based Microcapsules According to the Invention

The sensory evaluation was carried out as follows: The above-mentioned microcapsules were each incorporated into a fabric softener with a slurry concentration of 0.4 wt.-% (perfume Tomcap) and then washed. Loading the capsules with 17.5% perfume oil (+17.5% vegetable oil=total loading 35%) and a dosage of 0.4% capsule slurry results in a dosage of pure perfume oil of 0.07% perfume oil in the fabric softener for comparison. 30 g of fabric softener was used for 2 kg of laundry including the Terry Towels. The washing instructions were as follows: The wash item including the Terry-Towels (cotton towel) was placed in the washing machine and the fabric softener was placed in the fabric softener compartment. The washing program “Express 20; 900 rpm was started. The Terry Towels were then left to dry overnight at room temperature.

16 test subjects assessed the fragrance intensity of the Terry-Towels after washing by testing them in pairs against a corresponding level of free perfume oil in the fabric softener on a scale from 1 (no odor) to 9 (very strong odor).

The fragrance was released in three steps. The first step describes the smelling of an untreated cloth. The second step describes the smelling of a lightly kneaded cloth; for this purpose, the cloth was subjected to slight mechanical stress by moving it back and forth between the hands several times, causing the capsules to break. The third step describes the smelling after the cloths were rubbed vigorously, causing the capsules to break. The fragrance intensity was evaluated after each step.

The results of the sensory evaluation are shown in Table 8.

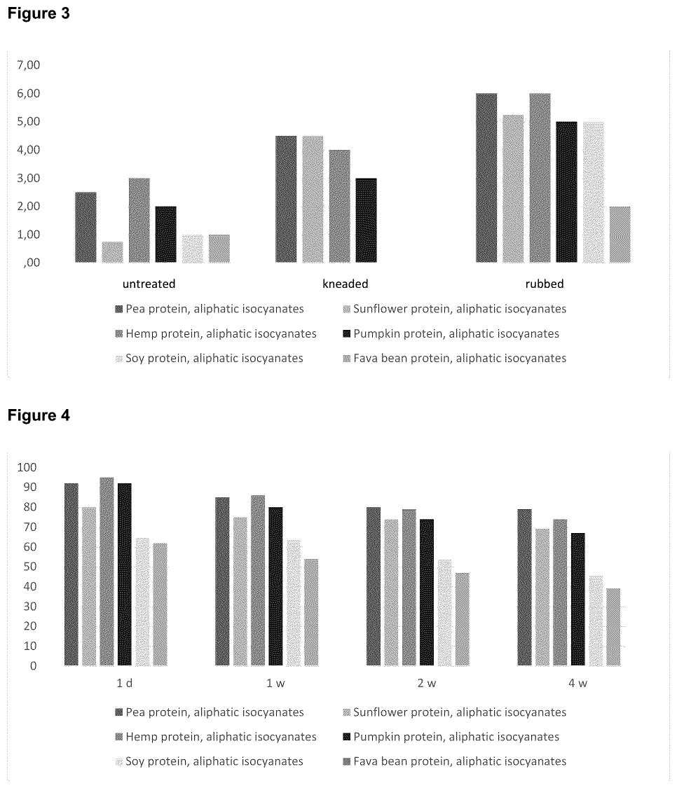

In general, it should be noted that preferably used plant proteins, such as pea protein, pumpkin protein, sunflower protein and hemp protein, each enable the provision of plant protein-based microcapsules according to the invention, whereby good sensory properties can always be achieved (see also FIG. 3). In accordance with the core idea of the invention, the sensory properties of those plant protein-based microcapsules are better when aliphatic polyisocyanates are used as crosslinking agents (see Table 8, lines 2 and 8).

TABLE 8

| | Sensory properties | | |
|---|---|---|---|
| Capsule type | untreated | kneaded | rubbed |
| Pea protein, aliphatic | 2.5 | 4.5 | 6.0 |
| Isocyanates | | | |
| Pea protein, mixture | 2.7 | — | 3.8 |
| Aromatic and aliphatic isocyanates | | | |
| Sunflower protein, aliphatic | 0.8 | 4.5 | 5.3 |
| Isocyanates | | | |
| Sunflower protein, mixture | — | — | — |
| Aromatic and aliphatic isocyanates | | | |
| Hemp protein, aliphatic isocyanates | 3.0 | 4.0 | 6.0 |
| Hemp protein, blend aromatic | — | — | — |
| and aliphatic isocyanates | | | |
| Pumpkin protein, aliphatic isocyanates | 2.0 | 3.0 | 5.0 |
| Pumpkin protein, mixture | 1.0 | 2.0 | 3.0 |
| Aromatic and aliphatic isocyanates | | | |

The results according to Table 8 are also shown in FIG. 2.

In accordance with the above, it was surprisingly found that the addition of hyaluronic acid in the preparation of the plant protein-based microcapsules according to the invention with at least one aliphatic polyisocyanate as crosslinker has a positive effect on the sensory properties. It was particularly surprising to note that good sensory properties can be achieved by adding hyaluronic acid and that the amount of isocyanate can be reduced at the same time (see Table 9, lines 5 and 6 below). By reducing the amount of polyisocyanate in this way, biodegradability can be improved.

TABLE 9

| | Sensory properties | | |
|---|---|---|---|
| Capsule type | untreated | kneaded | grated |
| Pea protein, 28% isocyanates | 0.0 | 1.5 | 2.5 |
| Pea protein, 28% isocyanates with | 2.3 | 3.0 | 5.3 |
| hyaluronic acid | | | |
| Pea protein, 23% isocyanates | 1.5 | 3.0 | 4.2 |
| Pea protein, 23% isocyanates with | 2.5 | 3.5 | 5.0 |
| hyaluronic acid | | | |
| Pea protein, 19% isocyanates | 0.0 | 1.5 | 2.3 |
| Pea protein, 19% isocyanates with | 1.0 | 2.0 | 4.0 |
| hyaluronic acid | | | |

The results according to Table 9 are also shown in FIG. 6.

This was surprisingly found in connection with the addition of glycerol and hyaluronic acid (see Table 10, lines 8 and 9).

In accordance with the above, it was surprisingly found that the addition of hyaluronic acid and glycerol in the preparation of the plant protein-based microcapsules according to the invention with at least one aliphatic polyisocyanate as crosslinker has a positive effect on the sensory properties. It was particularly surprising to note that good sensory properties can be achieved by adding hyaluronic acid and glycerol and that the amount of isocyanate can be reduced at the same time (see Table 10 below, lines 8 and 9). Such a reduction in polyisocyanate can improve biodegradability.

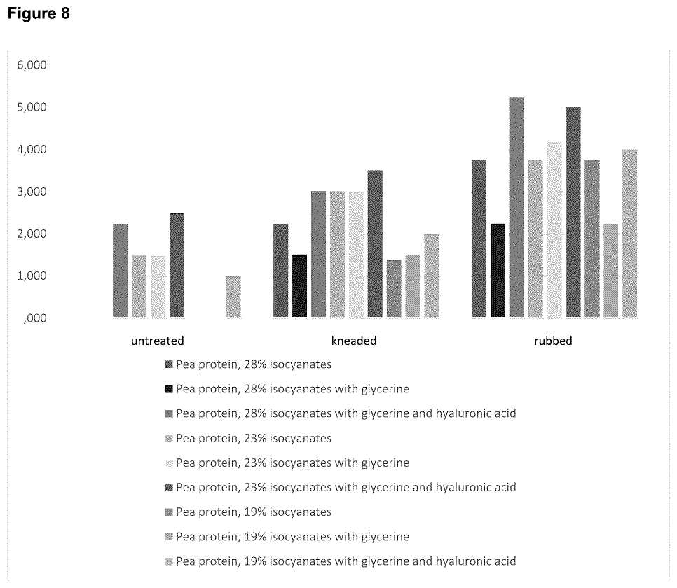

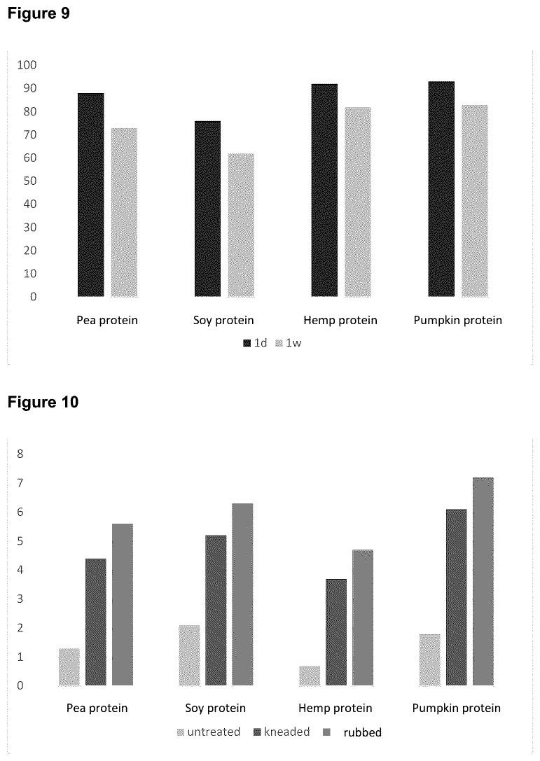

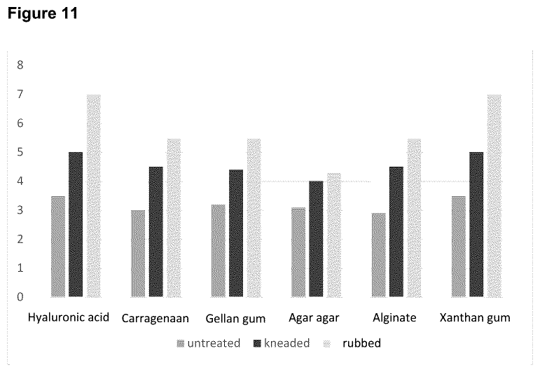

The results according to Table 10 are also shown in FIG. 8.

TABLE 10

| | Sensory properties | | |
|---|---|---|---|
| Capsule type | untreated | kneaded | grated |
| Pea protein, 28% isocyanates | 0.00 | 2.25 | 3.75 |
| Pea protein, 23% isocyanates | 1.50 | 3.00 | 3.75 |
| Pea protein, 19% isocyanates | 0.00 | 1.38 | 3.75 |
| Pea protein, 28% isocyanates | 0.00 | 1.50 | 2.25 |
| with glycerine | | | |
| Pea protein, 23% isocyanates | 1.50 | 3.00 | 4.20 |
| with glycerine | | | |
| Pea protein, 19% isocyanates | 0.00 | 1.50 | 2.25 |
| with glycerine | | | |
| Pea protein, 28% isocyanates | 2.3 | 3.0 | 5.3 |
| with glycerine and hyaluronic acid | | | |
| Pea protein, 23% isocyanates | 2.5 | 3.5 | 5.0 |
| with glycerine and hyaluronic acid | | | |
| Pea protein, 19% isocyanates | 1.0 | 2.0 | 4.0 |
| with glycerine and hyaluronic acid | | | |

### Example 3: Further Results on the Stability of the Plant Protein-Based Microcapsules According to the Invention in Use and Further Sensory Evaluations of the Plant Protein-Based Microcapsules According to the Invention

However, plant protein-based microcapsules according to the invention with very good stability properties and very good sensory properties are not limited to pea protein. Rather, it was surprisingly found that the microcapsules produced using soy protein, hemp protein or pumpkin protein instead of pea protein also have very good sensory properties and very good stability properties, which can even exceed the stabilities, as can be seen from Tables 11 and 12 shown below.

TABLE 11

| | Stability in fabric | Stability in fabric |
|---|---|---|
| | softener @ | softener @ 40° C. [%], |
| Plant protein | 40° C. [%], 1 d | 1 w |
| Pea protein (reference) | 88 | 73 |
| Soy protein (instead of pea | 76 | 62 |
| protein) | | |
| Hemp protein (instead of pea | 92 | 82 |
| protein) | | |
| Pumpkin protein (instead of | 93 | 83 |
| pea protein) | | |

The results according to Table 11 are also shown in FIG. 9.

However, plant protein-based microcapsules according to the invention with very good stability properties and very good sensory properties are not limited to pea protein. Rather, it was surprisingly found that the microcapsules produced using soy protein, hemp protein or pumpkin protein instead of pea protein also have very good sensory properties and very good stability properties, which even exceed the stability properties, as can be seen from Tables 11 and 12 below.

TABLE 12

| | Sensory properties | | |
|---|---|---|---|
| Capsule type | untreated | kneaded | grated |
| Pea protein (reference) | 1.30 | 4.40 | 5.60 |
| Soy protein (instead of pea protein) | 2.10 | 5.20 | 6.30 |
| Hemp protein (instead of pea protein) | 0.70 | 3.70 | 4.70 |
| Pumpkin protein (instead of pea protein) | 1.80 | 6.10 | 7.20 |

The results according to Table 12 are also shown in FIG. 10.

It was surprisingly found that microcapsules with particularly good sensory properties can be obtained if the at least one first polysaccharide and/or the at least one further polysaccharide is selected from the group consisting of hyaluronic acid, carrageenan, gellan gum, agar agar, alginate, xanthan gum, as can be seen from the following table.

TABLE 13

| | Sensory properties | | |
|---|---|---|---|
| Added polysaccharide | untreated | kneaded | grated |
| Hyaluronic acid | 3.50 | 5.00 | 7.00 |
| Carragenaan | 3.00 | 4.50 | 5.50 |
| Gellan gum | 3.20 | 4.40 | 5.50 |
| Agar agar | 3.10 | 4.00 | 4.30 |
| Alginate | 2.90 | 4.50 | 5.50 |
| Xanthan gum | 3.50 | 5.00 | 7.00 |

The results according to Table 13 are also shown in FIG. 11.

1. A process for preparing a plant protein-based microcapsule comprising the following steps in this order:
(i) providing an internal non-aqueous phase comprising at least one aliphatic polyisocyanate as a crosslinking agent and at least one hydrophobic active ingredient and optionally at least one further crosslinking agent;
(ii) providing an external aqueous phase comprising at least one plant protein
and optionally at least one first polysaccharide and/or at least one further crosslinking agent and/or at least one polyhydroxyphenol and/or at least one protective colloid, and further optionally adjusting the pH of the aqueous phase to a pH below the isoelectric point of the plant protein;
(iii) emulsifying or dispersing the internal non-aqueous phase in the external aqueous phase, optionally in the presence of at least one stabilizer and/or at least one emulsifier, to obtain an oil-in-water emulsion or dispersion;
(iv) optionally adding at least one further polysaccharide and/or at least one further plant protein;
(v) carrying out a first cross-linking to obtain a microcapsule slurry;
(vi) curing the microcapsule slurry at a temperature of at least 60° C. and optionally adding a further polysaccharide and/or a further plant protein;
(vii) cooling and optionally carrying out a further crosslinking by addition of at least one further crosslinking agent; and
(viii) optionally, separating the microcapsules from the microcapsule slurry and, if necessary, drying the microcapsules or adjusting the viscosity of the microcapsule slurry by adding at least one thickening agent.

2. The process according to claim 1, wherein at least one of the aliphatic polyisocyanates or the one aliphatic polyisocyanate is a cycloaliphatic polyisocyanate having two or more isocyanate groups.

3. The process according to claim 1, wherein the total amount of polyisocyanate, based on the amount of wall former, is 10 to 70% by weight, or wherein the total amount of polyisocyanates used as crosslinking agents together is 0.5% to 4%, based on the total amount of hydrophobic active ingredient used.

4. The process according to claim 1, wherein the polysaccharide or the at least one further polysaccharide is hyaluronic acid or wherein the polyhydroxyphenol is a tannin.

5. The process according to claim 4, wherein the amount of hyaluronic acid relative to the amount of wall former is 1 to 15 percent by weight.

6. The process according to claim 1, wherein in step (i) an internal non-aqueous phase is provided comprising
(a) an aliphatic polyisocyanate as a crosslinking agent and a cycloaliphatic polyisocyanate as crosslinking agent, or
(b) two different aliphatic polyisocyanates as crosslinking agents wherein the aliphatic polyisocyanate and the cycloaliphatic polyisocyanate, or the two different aliphatic polyisocyanates, are used in a respective molar ratio of 85:15 to 15:85.

7. (canceled)

8. The process according to claim 1, wherein in step (i) an internal non-aqueous phase is provided comprising three crosslinking agents, wherein the three crosslinking agents are aliphatic or cycloaliphatic polyisocyanates different from each other, wherein at least one cycloaliphatic polyisocyanate and at least one aliphatic polyisocyanate are present, and wherein the three polyisocyanates each are used in amounts of 20% to 60% based on the total weight of the three polyisocyanates.

9. The process according to claim 1, wherein in step (i) at least one further crosslinking agent and/or in step (ii) and/or in step (vii) a further crosslinking agent is added, wherein the at least one further crosslinking agent and/or the further crosslinking agent is selected from the group consisting of transglutaminase, peroxidase, secondary plant substances selected from the group consisting of polyphenols and polyhydroxyphenols, and mixtures of two or more of the aforementioned crosslinking agents.

10. The process according to claim 1, wherein the at least one hydrophobic active ingredient is selected from the group consisting of fragrances or odorants, flavorings, cooling agents, TRPV1 and TRPV3 modulators, substances that produce a sharp taste or a warmth or heat sensation on the skin or mucous membranes or a fizzing or tingling sensation in the mouth or throat or active ingredients with pungent or acrid or astringent effects, pesticides, biocides, insecticides, a substance from the group of repellents, food additives, cosmetic active substances, pharmaceutical active substances, agrochemicals, dyes, colorants, dye precursors; luminous paint, optical brighteners, solvents, waxes, silicone oils, lubricants, printing coatings for paper and mixtures of two or more of the aforementioned active ingredients.

11. The process according to claim 1, wherein the at least one first polysaccharide and/or the at least one further polysaccharide is selected from the group consisting of
indigestible fibers and dietary fibers;
starches and starch derivatives
sugar alcohols;
gellan; glucose,
glycosaminoglycans;
and mixtures of the aforementioned polysaccharides.

12. The process according to claim 1, wherein the at least one plant protein or the at least one further plant protein is selected from the group consisting of protein isolates, plant proteins in the form of fractions, and partial or complete hydrolysates or intermediates produced by physico-chemical processes or fermentative or enzymatic treatment of the proteins.

13. The process according to claim 1, wherein the at least one protective colloid is selected from the group consisting of
diols
polyols,
polyvinylpyrrolidone, maleic acid vinyl copolymers, sodium lignosulfonates, maleic acid anhydride/styrene copolymers, ethylene/maleic acid anhydride copolymers, copolymers of ethylene oxide, propylene oxide and acid esters of polyethoxylated sorbitol, sodium dodecyl sulfate,
vegetable polymers,
and mixtures of the aforementioned compounds;
and/or wherein the protective colloid is used in combination with starch.

14. The process according to claim 1, wherein in step (v) the first crosslinking is carried out to obtain a microcapsule slurry by adding at least one catalyst; wherein the at least one catalyst is selected from the group consisting of diazobicyclo[2.2.2]octane (DABCO), bismuth catalyst, tin catalyst, and mixtures of two or more of the aforementioned catalysts.

15. The process according to claim 1, wherein the total amount of crosslinking agent added as polyisocyanates in step (i) is 0.5% to 4% based on the total amount of hydrophobic active ingredient used in step (i).

16. (canceled)

17. A plant protein-based microcapsule comprising
(a) a core comprising at least one hydrophobic active ingredient;
(b) a capsule shell comprising a crosslinking matrix or crosslinking units of at least one plant protein and at least one aliphatic polyisocyanate as a crosslinking agent and optionally at least one polysaccharide, and optionally at least one protective colloid, and/or optionally at least one further crosslinking agent.

18. The microcapsule according to claim 17, wherein the capsule shell comprises: a crosslinking matrix or crosslinking units of a polymerization and/or crosslinking of at least one plant protein with a mixture of at least two different crosslinking agents, and a crosslinking matrix or crosslinking units of a polymerization and/or crosslinking of at least one polysaccharide, with a mixture of the at least two different crosslinking agents.

19. The microcapsule according to claim 17, wherein the at least two crosslinking agents are each different aliphatic or cycloaliphatic polyisocyanates having two or more isocyanate groups.

20. The microcapsule according to claim 17, wherein the total amount of the polyisocyanates used as crosslinking agents together is 0.5% to 4% based on the total amount of hydrophobic active ingredient used.

21. The microcapsule according to claim 17, wherein the microcapsule comprises hyaluronic acid.

22. (canceled)

23. Household products, fabric care products, laundry detergents, fabric softeners, cleaning agents, scent boosters and fragrance enhancers, cosmetics, personal care products, perfume compositions, agricultural products or pharmaceutical products, comprising a microcapsule according to claim 17.
